(19) United States
(12) Patent Application Publication
(10) Pub. No.: US 2024/0324606 A1
(43) Pub. Date: Oct. 3, 2024

(54) ARTHROPOD CONTROL PRODUCTS

(71) Applicant: Firmenich SA, Satigny (CH)

(72) Inventors: Vincent HARRACA, Satigny (CH); Wolfgang FIEBER, Satigny (CH); Pascal BEAUSSOUBRE, Satigny (CH)

(21) Appl. No.: 18/293,854

(22) PCT Filed: Sep. 28, 2022
(86) PCT No.: PCT/EP2022/077028

§ 371 Date: Jan. 31, 2024

(30) Foreign Application Priority Data
Sep. 29, 2021 (EP) 21199748.1

(51) Int. Cl. A01N 65/44; A01N 25/04; A01N 25/22; A01N 31/02; A01N 53/00; A01N 65/22; A01N 65/24; A01N 65/28; A01P 7/04

(52) U.S. Cl. CPC A01N 65/44; A01N 25/04; A01N 25/22; A01N 31/02; A01N 53/00; A01N 65/22; A01N 65/24; A01N 65/28; A01P 7/04

(57) ABSTRACT

Described herein is an oil-in-water emulsion including a solid lipid material in combination with an oil-soluble active arthropod control agent. Also described herein are a process for preparing the emulsion and consumer articles including said the emulsion.

### TECHNICAL FIELD

The present invention relates to the field of arthropod control products. More specifically, the invention concerns an oil-in-water emulsion comprising a solid lipid material in combination with an oil-soluble arthropod control agent. The present invention also relates to the use of said emulsion as an arthropod control product and consumer articles comprising said emulsion.

### BACKGROUND OF THE INVENTION

Many mammals, including humans, suffer from the action of arthropods since they represent target host organisms. Some arthropods, for example mosquitoes and ticks, are not desirable for vertebrates such as mammals and in particular human subjects, as they bite and, consequently, cause itching, transmission of diseases and/or germs or may be the cause for other diseases and/or conditions.

Arthropod control products include active substances which when applied to skin or clothing discourage arthropods from landing or climbing on that surface. Arthropod control agents help preventing and controlling the outbreak of arthropod-borne diseases, such as malaria, etc. However, when applied to the skin or clothing such compositions are intended only to discourage the arthropod interacting with the target host organism. They provide only a short time remedy since the arthropod control composition can degrade over time or in other ways lose their effectiveness. Moreover, since known arthropod control compositions typically are applied to a target host organism, then the arthropod must already be in close proximity to its target before it gets repelled, which increases the risk of the arthropod overcoming the effectiveness of the arthropod control composition and reaching its target.

Furthermore, some of the known arthropod controlling agents and compositions have certain drawbacks, for example negative olfactive properties such as no or bad smell, or low lastingness due to penetration through the surface to be protected on which it is applied, or in turn only weak arthropod controlling, in particular arthropod repelling properties.

Therefore, the development of arthropod products which can be applied to the arthropod itself or to areas in which the arthropod passages, in order to discourage engagement with its target host organism and at the same time having good long lastingness properties and good arthropod control performance, in particular arthropod repelling properties, remains a technical challenge.

### SUMMARY OF THE INVENTION

The present invention solves the above-mentioned problem by providing an oil-in-water emulsion comprising a solid lipid material used in combination with a oil-soluble active material that can be applied directly to a target arthropod or in an area of habitation of an arthropod.

A first object of the invention provides an oil-in-water emulsion comprising:
a dispersed oil phase comprising at least one solid lipid material and at least one oil-soluble arthropod control agent, and
a continuous aqueous phase comprising a stabilizer, wherein said emulsion is for use as an arthropod control product by application directly on a target arthropod and/or application to an area of habitation of an arthropod.

A second aspect of the invention provides a method for the preparation of an oil-in water emulsion for use as an arthropod control product, said process comprising the steps of:
(i) Dispersing an oil phase comprising at least one solid lipid material and at least one oil-soluble arthropod control agent into a continuous aqueous phase comprising a stabilizer to obtain an oil-in-water emulsion, at a temperature above the melting point of the solid lipid material;
(ii) Cooling the emulsion thus obtained to a temperature below the melting point of the solid lipid material.

A third object of the invention is a consumer article comprising the emulsion as defined above.

A fourth object of the invention is a method for controlling arthropod, the method comprising the steps of applying or diffusing the emulsion as defined above herein on the arthropod and/or in an area of habitation of an arthropod.

### FIGURES

FIG. 1: Images of Emulsion 1 (A) & Emulsion 2 (B) displaying the difference in droplet shape comprising the solid lipid material due to formulation process.

FIG. 2: Tracks of the movement of Tenebrio molitor in the arena with the lower half treated with 5% EO (A), water (B) or Emulsion 5 (C). The upper half was untreated and served as refuge.

FIG. 3: Microscopic images of different body parts of different arthropods, treated with different products or untreated. Tarsi of Periplaneta americana (L) untreated (A) or treated with Emulsion 2 (B) and Tenebrio molitor (L) elytron untreated (C) or treated with 0.5% GLf (D) or Emulsion 2 (E). Treatments were loaded with a fluorescent agent displaying the position of the active in the different figures.

### DETAILED DESCRIPTION OF THE INVENTION

Unless stated otherwise, percentages (%) are meant to designate a percentage by weight of a composition.

A first object of the invention is an oil-in-water emulsion comprising:
a dispersed oil phase comprising at least one solid lipid material and at least one oil-soluble arthropod control agent, and
a continuous aqueous phase comprising a stabilizer,

wherein said emulsion is for use as an arthropod control product by application directly on a target arthropod and/or application to an area of habitation of an arthropod.

In an embodiment of the invention the emulsion comprises more than 50% water, preferably, 60%, 65%, 68%, 70%, 73%, 75%, 80%, 85% or more.

In a preferred embodiment of the invention the emulsion comprises more than 70% water, preferably 73%, 75%, 80%, 85% or more.

In a further embodiment of the invention the emulsion comprises more than 0.01% arthropod control agent, preferably, 0.05%, 0.1%, 0.5%, 1%, 2%, 5%, 10%, 20%, 25% or more.

Oil-in-water emulsions are widely used in consumer products. They comprise typically a continuous aqueous phase and a dispersed oil phase containing active ingredients, such as for example pharmaceutical actives, emollients, essential oils, vitamins, pigments, or perfumes. A frequent usage of oil-in-water emulsions is the direct application onto skin in the form of lotions or creams. The targeted goals are penetration of actives through the skin, the deposition of actives on the skin or evaporation of actives from the skin.

However, until the present invention oil-in-water emulsions have not been used to deliver arthropod control agents by application directly on a target arthropod and/or application to an area of habitation of an arthropod. An advantage of the emulsion of the present invention is that the oil phase adheres to the cuticle of the arthropod thus aiding the delivery of the control agent.

The invention provides an emulsion with a high amount of water and a low amount of arthropod control agent relative to the other components of the emulsion. As can be appreciated, these features allow the emulsion to be prepared at low cost and in a sustainable manner compared to alternative embodiments in which more expensive and less sustainable components are used at high levels.

The emulsion of the invention comprises an oil phase comprising at least one solid lipid material. As shown in the accompanying examples this solid lipid material forms solid droplets in the emulsion and comprises arthropod control agent. The size of the solid droplets can vary (as shown in the accompanying examples and figures) which has the beneficial effect of allowing the arthropod control agent to diffuse out of the emulsion over a range of time.

The aspects of the present invention relate to the application of the emulsion of the invention directly on a target arthropod and/or application to an area of habitation of an arthropod.

By “directly on the target arthropod” we include where the emulsion is deliberately sprayed or applied onto the arthropod. This can be achieved by a user of the emulsion of the invention aiming a dispensing device directly at the target arthropod and dispensing the emulsion directly onto the arthropod or in such close proximity to the arthropod that it is inevitable at least some of the emulsion of the invention will be applied onto the arthropod.

By “application to an area of habitation of an arthropod” we include where the emulsion is deliberately sprayed or applied into an area where the arthropod routinely or periodically resides, such as a nest, breeding area, congregation area, or other such arthropod-suitable living areas, or to a location where it is anticipated that the arthropod will pass through during its typical living activities, such as moving around the boundary of its living area or between its living area and an expected food source.

According to an embodiment, the present invention does not use any magnetically polarized particles to adhere to the substrate.

A further preferred embodiment is wherein the emulsion and/or the arthropod control agent is not encapsulated.

Capsules contain by definition a capsule core (containing active arthropod control agent±adjuvants) and a capsule shell (physically separating the core from the surrounding medium). Encapsulated arthropod control products are known in the art.

It is important to note that the emulsion of the invention is not considered as encapsulated since it does not have a shell. The absence of a shell encapsulating the active material in the emulsion and/or the control agent of the invention is an important advantage over the prior art, since the solid droplets in the emulsion of the invention can readily bind to the exoskeleton of the targeted arthropod, allowing the diffusion of the arthropod control agent in proximity of the target exoskeleton and hence being unwittingly distributed by the targeted arthropod. Therefore, the arthropod can be used as a vehicle to distribute the arthropod control agent. Indeed, an arthropod exoskeleton is externally covered by a waxy layer placed above the cuticle comprised mainly of chitin that protects the insect against dehydration, microbial infection and physical injury. In addition, damaging or penetrating the cuticle is enhanced by a longer contact time and such property would be provided by the invention.

Binding the arthropod control agent to the exoskeleton of the targeted arthropod has the advantage of having the arthropod control agent being transported by the targeted arthropod. As a result, the arthropod control agent will be distributed to locations where application of such agents is difficult or impossible, for example hidden crevices. Moreover, in the case of gregarious or eusocial arthropods, since the arthropod carrying the arthropod control agent will seek to join peers, the arthropod control agent will be brought into the gathering and/or harboring site of this species. In addition to being relatively species specific, this will disorganize the targeted pest group e.g. increasing the likelihood of leaving the secure harboring site and being more at risk of predator or pesticide

This capability of using a wax coating layer to attach to a substrate is mentioned by US20170245493A1, but in their claim the coating layer is used to attach microencapsulated essential oil specifically to a non-woven fabric due to non-covalent association, hence teaching away from using a non-encapsulated emulsion composition of the present invention.

US20020179075A1 describes a pesticide delivery system that directly contacts the pest target, but here the system is made of a flexible material and is supposed to burst upon impact.

None of the prior art documents discussed herein contemplates the preparation of an emulsion composition of the invention, let alone an unencapsulated emulsion.

In a further embodiment of the invention is wherein the oil-soluble active ingredient has a Log P>0.5

A further embodiment of the invention is wherein the emulsion does not comprise alcohol, in particular ethanol.

### Emulsion:

The term “emulsion”, as used herein, denotes a mixture of two or more liquids that are normally immiscible (i.e. not soluble). In an emulsion, one liquid (the dispersed phase) is dispersed in the other (the continuous phase). The present invention is directed to oil-in-water emulsions comprising a continuous hydrophilic phase in which the oil-soluble phase is dispersed. According to the invention, the oil-soluble material is in the dispersed phase. However, one part of the oil-soluble material may also be present in the continuous phase depending on the polarity of the active ingredient.

According to one embodiment, the emulsion is a macroemulsion or a nanoemulsion. An emulsion according to the invention may be prepared by any method applying mechanical forces to emulsify the disperse phase droplets, preferably by mechanical mixing with a high shear blender, a colloidal mill, an impeller mixer, or by the use of a high-pressure homogenizer. Alternatively, such emulsions may also be prepared by ultrasound processing, by membrane emulsification, or by emulsification using microfluidic channels.

The emulsion can be in the form of a gel, preferably having a viscosity comprised between 1 mPa·s and 1 Pa·s, preferably between 1 mPa·s and 500 mPa·s, wherein the viscosity is measured at 25° C. with a shear rate of 100 s⁻¹. The flow viscosity was measured using a TA Instruments AR2000 rheometer (New Castle, DE, USA) with a cone-plate geometry.

It is important to note that the emulsion of the present invention is an “oil-in-water” emulsion and not a “water-in-oil” emulsion. An “oil-in-water” emulsion has oil droplets dispersed in a liquid, aqueous environment, which is important for the active ingredient(s) in the emulsion. In contrast, a “water-in-oil” emulsion has water droplets in a liquid oil environment which would alter the effectiveness of the active ingredient(s) in the emulsion.

### Solid Lipid Material:

The term “solid lipid material” used in the present invention refers to lipid components that are solid or in the form of a paste at room temperature. It includes glycerides and waxes. By contrast, the term “oil” used in the present invention refers to organic components that are liquid at room temperature.

The use of a solid lipid oil material means that the arthropod control agent is trapped in a matrix formed by the solid lipid. Such a composition reduces the diffusivity of the active arthropod control agent out of the matrix and slows down evaporation into the air and on arthropod surface improving its lastingness and therefore its action.

Additionally, the active agent trapped in the solid droplet core is also protected from harsh environmental conditions, decreasing the risk of degradation due for instance to light as it was demonstrated for instance by Nguyen et al. (2012, doi: 10.1002/ps.3268) with nanoparticles made of solid beeswax coated with chitosan.

According to an embodiment, the solid lipid oil material would bind to arthropods' cuticle made of hydrophobic substances, ensuring better contact and transport of the active agent along with the targeted arthropod. This adhesion process based on gravity and hydrophobicity is similar to naturally occurring adherence between entomopathogenic conidia and insect epicuticle (e.g. Boucias et al., 1988, 10.1128/aem.54.7.1795-1805.1988) allowing fixation and then dissemination.

According to an embodiment, the solid lipid material is chosen in the group consisting of vegetable fats and non-vegetal fats. In one embodiment the solid fat is a derivative of vegetable fatty acids and glycerol in the form of a triglyceride. In one particular embodiment the triglyceride is palm stearin.

According to an embodiment the solid lipid material is chosen in the group consisting of vegetal waxes and non-vegetal waxes. In one particular embodiment the non-vegetal wax is beeswax. In one particular embodiment the vegetal wax is carnauba wax or jojoba wax.

In a preferred embodiment of the invention the solid lipid material is natural biodegradable wax and not a non-natural wax. An advantage of this aspect of the invention is that the emulsion of the invention will have less impact on the environment since natural waxes are already known and used in consumer good.

Furthermore natural biodegradable waxes could help address any regulatory authority concerns which could arise in relation to the use of the emulsion of the invention.

According to an embodiment, the solid lipid material is chosen in the group consisting of beeswax, carnauba wax, palm stearin, jojoba wax and mixtures thereof.

According to an embodiment, the solid lipid material is not castor oil.

The amount of solid lipid material is preferably comprised between 0.5 to 50%, preferably between 1% and 10%, preferably, 1% and 5%, preferably around 1.5% by weight based on the total weight of the oil phase.

In a particular embodiment, due to the lipid composition of the solid droplet core containing the active agent, the arthropod control agent may be ingested by the targeted pest during grooming for instance increasing even more the efficacy of the arthropod control agent that would directly access the more permeable mucosal surface of the gustatory tract of the arthropod. In this invention, the composition and size of the lipid core containing the active agent would directly favor the passive ingestion by the arthropod.

### Oil-Soluble Active Material:

The oil-soluble material is a single material or a mixture of materials—which forms a two-phase dispersion when mixed with water.

A preferred embodiment of the invention is wherein the oil-soluble active material is soluble in oil and is defined with a Log P greater than 0.5, preferably greater than 1, more preferably greater than 1.2.

According to an embodiment, the oil soluble active material has a Log P greater than 0.5 and less than 8. According to another embodiment, the oil soluble active material has a Log P greater than 0.5 and less than 5. According to another embodiment, the oil soluble active material has a Log P greater than 0.5 and less than 3.

Log P is the common logarithm of estimated octanol-water partition coefficient, which is known as a measure of lipophilicity.

The Log P values of many compounds have been reported, for example, in the Pomona92 database, available from Daylight Chemical Information Systems, Inc. (Daylight CIS), Irvine, Calif., which also contains citations to the original literature. Log P values are most conveniently calculated by the “C LOG P” program, also available from Daylight CIS. This program also lists experimental log P values when they are available in the Pomona92 database. The “calculated log P” (c Log P) is determined by the fragment approach of Hansch and Leo (cf. A. Leo, in Comprehensive Medicinal Chemistry, Vol. 4, C. Hansch, P. G. Sammens, J. B. Taylor and C. A. Ramsden, Eds., p. 295, Pergamon Press, 1990). The fragment approach is based on the chemical structure of each perfume oil ingredient and takes into account the numbers and types of atoms, the atom connectivity, and chemical bonding. The c Log P values, which are the most reliable and widely used estimates for this physicochemical property, are preferably used in the selection of perfuming compounds which are useful in the present invention when no experimental Log P values are available. According to a preferred embodiment, the ratio of arthropod control agent to solid lipid material is 1:200, 1:150, 1:120, 1:100, 1:50 1:25, 1:10, 1:5, 1:1, 2:1, 3:1, 4:1, 5:1, 6:1, 7:1, 8:1, 9:1, 10:1 or more. Preferably the ratio of arthropod control agent to solid lipid material is 1:200, 1:150, 1:120, 1:100, 1:50 1:25, 1:10, 1:5, 1:1, 2:1, 3:1, 4:1, 5:1, 10:1. Even more preferably from 1:20 to 10:1. The experimental data below provides examples of favorable arthropod control agent to solid lipid material ratios.

According to a preferred embodiment, the arthropod control agent comprises at least one arthropod control agent.

As shown in the accompanying examples, the emulsion of the invention comprises at least one arthropod control agent, and that the agent imparts an arthropod control activity to the emulsion. While the term “long lasting” is subjective, in this context it is intended to mean that the arthropod control activity of the arthropod control agent persists for a longer time when prepared in the form of the emulsion of the invention than in an alternative form where the arthropod control agent is not present in the emulsion of the invention. Though not being intended to limit the scope of the invention, it is the opinion of the inventors of the present invention that the arthropod control agent is present in a stabilised form in the emulsion and is released in a more regulated manner to provide a longer lasting efficacy than other formulations in which the arthropod control agent is prepared or in a free form, i.e. not in any emulsion at all.

### Arthropod Control Agent:

The term “arthropod” has the normal meaning for a skilled person in the technical field. Arthropods include invertebrate animals, such as insects, arachnids, and crustaceans, that have a segmented body and jointed appendages. Arthropods usually have a chitinous exoskeleton molted at intervals, and a dorsal anterior brain connected to a ventral chain of ganglia.

Arthropods in the present invention's understanding relate to undesired arthropods, meaning that their presence in the air, on the surface of an article, the surface of a plant or the surface of a vertebrate, such as a human subject or other mammal, preferably human subject, is not desired. Preferably undesired arthropods are pest arthropods that impact plants and animals including humans, and pest arthropods that impact human articles such as building materials, clothes, foods, furniture and other lifestyle belongings. For example, thrips, aphids, beetles, moths, mealybugs, scales, wasps, hornets, ants, termites, cockroaches, silverfish, spiders, etc., even more preferably blood feeding arthropods that impact vertebrates, e.g. biting flies, bed bugs, kissing bugs, fleas, lice, mosquitoes, ticks, etc, are considered target arthropods for the purposes of this application.

The arthropod control agent should affect and allow the control of any arthropods when chosen by a person skilled in the art. In an embodiment of the invention, arthropods that have a propensity to aggregate, preferably if they are gregarious, are preferred targets. In a further embodiment of the invention, arthropods displaying eusociality such as arthropods belonging to the Hymenoptera (ants, hornets, wasps) or Isoptera (termites) families are preferred targets. Differences between these social behaviors are known by a person skilled in the art.

The reason why the presence of an arthropod is not desired might be that the arthropod's presence in the vicinity is unpleasant to a subject, the contact of an arthropod on an article transfers diseases and/or germs, the arthropod is competing with human activity for resources that must be protected, the arthropod is damaging or destroying human articles or the arthropod bites an organism and causes itching, the transmission of diseases and/or germs or the arthropod feeding may be the cause for other diseases and/or conditions.

The expression “control”, “arthropod control” or the like has the normal meaning for a skilled person in the technical field. “Controlling” in the context of the present invention defines the ability of an agent or an arthropod controlling composition according to the present invention to attract, deter, kill, weaken or repel an arthropod, preferably kill, deter or repel an arthropod and even more preferably repel or deter an arthropod, and also disrupting the arthropod community and impacting breeding.

However, a preferred embodiment of the invention is wherein the arthropod control agent does not kill the arthropod. An advantage of this embodiment of the invention is that the arthropod can be used as a vector to transfer or transmit the agent to other arthropods and/or transfer said agent to the nest or other such shelter of habitation and therefore expose other arthropods to the arthropod control agent.

“Attracting” according to the present invention defines the ability of an agent to increase or encourage contact or the presence of an arthropod, of the same species or not, at the arthropod attractant source, such as in the air, on the surface of a shelter or a nest or another substrates on which are gathering or not these arthropods or on the surface of another arthropod to which the arthropod attractant agent or composition has been applied to.

“Deterring” according to the present invention defines the ability of arthropod control agent to minimize, reduce, discourage or prevent contact or the presence of an arthropod at the arthropod deterrent source, such as in the air, on the surface of a shelter or a nest or other substrates on which are gathering or not these arthropods or on the surface of another arthropod to which the arthropod deterrent agent or composition has been applied to. Typically, the deterrent effect is shown when used as deterrent hindering an arthropod from resting or aggregate with peers after an initial application of the deterrent agent or composition on the arthropod.

“Killing” according to the present invention defines the ability of arthropod control agent killing composition according to the present invention to kill an arthropod at the arthropod killing source, such as in the air, on the surface of shelter or a nest or other substrates on which are gathering or not these arthropods or on the surface of another arthropod to which the arthropod killing agent or composition has been applied to.

“Weaken” according to the present invention defines the ability of an agent weakening composition according to the present invention to diminish the resistance of an arthropod by lowering arthropods' chance of survival by intensifying the rate of desiccation, increasing the success of entomopathogenic or predators attacks, lowering the recognition by peers at the arthropod weakening source, such as in the air, on the surface of a shelter or a nest or another substrates on which are gathering or not these arthropods or on the surface of another arthropod to which the arthropod weakening agent or composition has been applied to.

“Repellency” according to the present invention defines the ability of arthropod control agent to minimize, reduce, discourage or prevent approach or presence of an arthropod at the arthropod repellent source, such as in the air, on the surface of shelter or a nest or other substrates on which are gathering or not these arthropods or on the surface of another arthropod to which the arthropod repellent agent or composition has been applied to.

Additional ingredients can be used in combination with an arthropod control agent. Non-limiting examples of such ingredients include a perfume, malodour counteracting, bactericide, fungicide, pharmaceutical or agrochemical ingredient, microbial agent, sanitizing agent, and mixture thereof.

According to a particular embodiment, the arthropod control agent is used in combination with substances which together improve, enhance or modify the delivery of the agent, such as precursors, emulsions or dispersions, as well as combinations which impart an additional benefit beyond that of modifying or imparting an effect, such as long-lasting, blooming, malodor counteraction, antimicrobial effect or microbial stability or microbial development.

The nature and type of the arthropod control agent that can be present in the oil-soluble internal phase do not warrant a more detailed description here, which in any case would not be exhaustive, the skilled person being able to select them on the basis of its general knowledge and according to the intended use or application.

According to an embodiment, the oil-soluble active agent is an arthropod control agent or a mixture of arthropod control agents.

According to a particular embodiment, the arthropod control agent is chosen in the group consisting of ethyl 3-(acetyl(butyl)amino)propanoate (IR3535*), N,N-diethyl-3-methylbenzamide (DEET), p-menthane-3,8-diol (PMD), Eucalyptus citriodora oil, Citronella spp. oil, sec-butyl 2-(2-hydroxyethyl)piperidine-1-carboxylate (picaridin), vanillin, Castor oil, Cedarwood oil, Cinnamon oil, citronellal, Clove oil, Corn oil, Cornmint, Cornmint oil, Cottonseed oil, 4-Allyl-2-methoxyphenol (Eugenol), Garlic oil, (2E)-3,7-Dimethylocta-2,6-dien-1-ol (Geraniol), Geranium oil, Lemongrass oil, Linseed oil, Peppermint, Peppermint oil, 2-Phenylethyl propionate, Rosemary oil, Sesame oil, Soybean oil, Spearmint, Spearmint oil, Thyme oil, Mint, Mint oil, Pepper extract, Wintergreen oil, Lavender oil, Lavandula hybrida ext., Lavandin oil, Lemon oil, Margosa extract, Mentha arvensis ext., Metofluthrin, Nonanoic acid, Pyrethrins and Pyrethroids, 2,3,4,5-bis(butyl-2-ene)tetrahydrofurfural (MGK Repellent 11), cineole, cinnamaldehyde, citral, citronellol, coumarin, dibutyl phthalate, diethyl phthalate, dimethyl anthranilate, dimethyl phthalate, ethyl vanillin, Eucalyptus oil, nootkatone, delta-octalactone, delta-nonalactone, delta-decalactone, delta-undecalactone, delta-dodecalactone, gamma-octalactone, gamma-nonalactone, gamma-decalactone, gamma-undecalactone, gamma-dodecalactone, hydroxy citronellal, lime oil, limonene, linalool, methyl anthranilate, Mint spicata, myrcene, Neem oil, sabinene, β-caryophyllene, (1H-indol-2-yl)acetic acid, anethole, Anise oil, Basil oil, Bay oil, camphor, ethyl salicylate, Evergreen oils, Pine oil, tetramethrin, allethrin, (RS)-α-cyano-3phenoxybenzyl-(1RS)-cis, cypermethrin, prallethrin, acetamiprid, azadirachtin, bendiocarb, bifenthrin, chlorpyrifos, deltamethrin, diazinon, dichlorvos, fipronil, imidacloprid, linalool, malathion, Margosa extract, nicotine, permethrin, rotenone, S-methoprene, spinosad (Spinosyn A), spinosyn D, transfluthrin, anisic alcohol, octahydrocoumarin, (+−)-2,5-dimethyl-2-indanmethanol, 4,4A,5,9B-tetrahydro-indeno[1,2-D]-1,3-dioxin, 2,4-dimethyl-4,4a,5,9b-tetrahydroindeno[1,2-d][1,3]dioxine, entomopathogenic bacterias such as Bacillaceae, Burkholderia, Chromobacterium, Pseudomonas, Saccharopolyspora, Serratia, Streptomyces, Yersinia, Xenorhabdus and Photorhabdus species, entomopathogenic fungi such as Metarhizium anisopliae, Beauveria bassiana, Hirsutella, Isaria, Lecanicillium, Paecilomyces and Verticillium species, entomopathogenic virus, entomopathogenic nematodes such as Steinernema and Heterorhabditis species, yeasts, specifically designed RNAi, enzymes such as chitinase, semiochemicals or pheromones such as stimuli eliciting sexual attraction (e.g. periplanetone B and gentisyl quinone isovalerate of the cockroaches Periplaneta americana (L) and Blattella germanica (L) respectively, (E,Z,Z)-3,8,11-Tetradecatrienyl acetate of the tomato leaf miner Tuta absoluta (Meyrick)) or aggregation (e.g. verbenone used by the bark beetles like Dendroctonus ponderosae (Hopkins) as an anti-aggregation pheromone or Z,E-alpha-farnesene used by the fire ants Atta geminata (Fabricius) to recruit and as an orientation pheromone) or identification of species (e.g. olefins and other hydrocarbons of termites such as Cryptotermes brevis (Walker), C. cynocephalus (Light), Procryptotermes corniceps (Snyder) or Neotermes connexus (Snyder)) or identification of colonies or nestmates (specific components of the cuticular hydrocarbon profile by wasps such as Polistes dominulus (Christ)) or information on development stage (e.g. oxo-aldehydes of the bed bugs Cimex lectularius (L)) or a message (such as E-β-farnesene and dimethylpyrazine used as alarm pheromones by aphids such as Phorodon humuli (Schrank) and the fire ants A. geminate (F) respectively), and mixtures thereof.

The use of biopesticide such as entomopathogenic bacteria, fungi, viruses or nematodes are well known to the person skilled in the art, see Kumar et al. (2021, doi: 10.3390/plants10061185), providing a review of different active agents.

The use of RNAi allows to mediate the silencing of specific targeted genes, blocking the expression of crucial proteins for the development of the targeted pest. Such techniques are known in the art and have the advantage of having a very specific impact on targeted pests, avoiding side effects on non-target organisms and getting around resistance.

The use of specific enzymes such as the chitinase in the emulsion of the invention would weaken the exoskeleton of the targeted arthropod, making it more prone to the risk of dehydration or attack by active agents such as entomopathogens or chemical substances.

The use of specific compounds such as pheromones would allow to greatly disturb communication between individuals. We can contemplate changing the hydrocarbon cuticular signature of insects causing a non-recognition by their kin or peers and therefore a rejection or an auto-destruction of the community in social insects (e.g. ants, termites, wasps). The solid droplet core allowing a transfer of the arthropod control agent by contact between cuticles, these new hydrocarbon cuticular signatures may rapidly spread within the colony, greatly disturbing colony structure. Similarly, spreading pheromones onto an insect would change is status among his peers. For instance, a cockroach carrying sexual pheromone due to presence of solid droplet core on his exoskeleton would attract more mating male partners than needed whether it is a male or a female. Similarly, as an example, spreading solid droplet core loaded with nymphs' specific pheromones onto adult bed bug would render them unattractive for sexual partner, decreasing the reproductive fitness of the population. In case of habituation as a resistance phenomenon, finding a sexual partner without using species specific pheromone will become more hazardous and even dangerous in the case of bedbugs due to their traumatic insemination methods. In both cases, targeted population of pests would be decreased due to lower mating fitness and success. In addition, spraying an arthropod or an area of habitation of an arthropod with an alarm pheromone or specifically chosen semiochemicals would cause nestmates to spend time and resources defending the colony against inexistent enemies. Similarly, spraying an arthropod or an area of habitation of an arthropod with a recruitment or an orientation pheromone would provoke the creation of fake tracks and therefore again waste time and energy of nestmates to look for inexistent resource.

In a preferred embodiment of the invention the arthropod control agent is liquid. According to such particular embodiment, the arthropod control agent is chosen in the group consisting of ethyl 3-(acetyl(butyl)amino)propanoate (IR3535*), N,N-diethyl-3-methylbenzamide (DEET), p-menthane-3,8-diol (PMD), Eucalyptus citriodora oil, Citronella spp. oil, sec-butyl 2-(2-hydroxyethyl)piperidine-1-carboxylate (picaridin), vanillin, Castor oil, Cedarwood oil, Cinnamon oil, citronellal, Clove oil, Corn oil, Cornmint, Cornmint oil, Cottonseed oil, 4-Allyl-2-methoxyphenol (Eugenol), Garlic oil, (2E)-3,7-Dimethylocta-2,6-dien-1-ol (Geraniol), Geranium oil, Lemongrass oil, Linseed oil, Peppermint, Peppermint oil, 2-Phenylethyl propionate, Rosemary oil, Sesame oil, Soybean oil, Spearmint, Spearmint oil, Thyme oil, Mint, Mint oil, Pepper extract, Wintergreen oil, Lavender oil, Lavandula hybrida ext., Lavandin oil, Lemon oil, Margosa extract, Mentha arvensis ext., Metofluthrin, Nonanoic acid, Pyrethrins and Pyrethroids, 2,3,4,5-bis(butyl-2-ene)tetrahydrofurfural (MGK Repellent 11), cineole, cinnamaldehyde, citral, citronellol, coumarin, dibutyl phthalate, diethyl phthalate, dimethyl anthranilate, dimethyl phthalate, ethyl vanillin, Eucalyptus oil, nootkatone, delta-octalactone, delta-nonalactone, delta-decalactone, delta-undecalactone, delta-dodecalactone, gamma-octalactone, gamma-nonalactone, gamma-decalactone, gamma-undecalactone, gamma-dodecalactone, hydroxy citronellal, lime oil, limonene, linalool, methyl anthranilate, Mint spicata, myrcene, Neem oil, sabinene, β-caryophyllene, (1H-indol-2-yl)acetic acid, anethole, Anise oil, Basil oil, Bay oil, camphor, ethyl salicylate, Evergreen oils, Pine oil, tetramethrin, allethrin, (RS)-α-cyano-3phenoxybenzyl-(1RS)-cis, cypermethrin, prallethrin, acetamiprid, azadirachtin, bendiocarb, bifenthrin, chlorpyrifos, deltamethrin, diazinon, dichlorvos, fipronil, imidacloprid, linalool, malathion, Margosa extract, nicotine, permethrin, rotenone, S-methoprene, spinosad (Spinosyn A), spinosyn D, transfluthrin, anisic alcohol, octahydrocoumarin, (+−)-2,5-dimethyl-2-indanmethanol, 4,4A,5,9B-tetrahydro-indeno[1,2-D]-1,3-dioxin, 2,4-dimethyl-4,4a,5,9b-tetrahydroindeno[1,2-d][1,3]dioxine, enzymes such as chitinase, semiochemicals or pheromones such as stimuli eliciting sexual attraction (e.g. periplanetone B and gentisyl quinone isovalerate of the cockroaches Periplaneta americana (L) and Blattella germanica (L) respectively, (E,Z,Z)-3,8,11-Tetradecatrienyl acetate of the tomato leaf miner Tuta absoluta (Meyrick)) or aggregation (e.g. verbenone used by the bark beetles like Dendroctonus ponderosae (Hopkins) as an anti-aggregation pheromone or Z,E-alpha-farnesene used by the fire ants Atta geminata (Fabricius) to recruit and as an orientation pheromone) or identification of species (e.g. olefins and other hydrocarbons of termites such as Cryptotermes brevis (Walker), C. cynocephalus (Light), Procryptotermes corniceps (Snyder) or Neotermes connexus (Snyder)) or identification of colonies or nestmates (specific components of the cuticular hydrocarbon profile by wasps such as Polistes dominulus (Christ)) or information on development stage (e.g. oxo-aldehydes of the bed bugs Cimex lectularius (L)) or a message (such as E-β-farnesene and dimethylpyrazine used as alarm pheromones by aphids such as Phorodon humuli (Schrank) and the fire ants A. geminate (F) respectively), and mixtures thereof.

A preferred embodiment of the invention is wherein the arthropod control agent is not N,N-diethyl-3-methylbenzamide (DEET).

A preferred embodiment of the invention is wherein the arthropod control agent is not ethyl 3-(acetyl(butyl)amino)propanoate (IR3535*).

In addition to the oil-soluble active material, the continuous phase can comprise a hydrophilic active agent dispersed or solubilized, preferably chosen in the group consisting of dried blood, lauryl sulfate, malic acid, Potassium (2E,4E)-hexa-2,4-dienoate, putrescent whole egg solids, sodium chloride, sulfuric acid monododecyl ester, sodium salt, zinc, boric acid, citric acid, maltodextrin, silicium dioxide, and mixtures thereof.

For the purpose of the present invention, additional ingredients entering inside the agent composition also includes combination of substances which together protect, improve, enhance or modify the delivery of the agent, such as compound precursors, emulsions or dispersions, as well as combinations which impart an additional benefit beyond that of modifying or imparting an odor, such as long-lasting, blooming, malodour counteraction, antimicrobial effect, microbial stability, arthropods control, or microbial development.

The nature and type of the additional ingredients present in the oil phase do not warrant a more detailed description here, which in any case would not be exhaustive, the skilled person being able to select them on the basis of its general knowledge and according to intended use or application and the desired organoleptic effect. In general terms, these additional ingredients belong to chemical classes as varied as alcohols, aldehydes, ketones, esters, ethers, acetates, nitriles, terpenoids, nitrogenous or sulphurous heterocyclic compounds and essential oils, and can be of natural or synthetic origin. Many of these co-ingredients are in any case well known in the art to the skilled person, or in other works of a similar nature, as well as in the abundant patent literature in the field of perfumery, pheromones, semiochemicals and entomopathogenic biocontrol agents. It is also understood that said ingredients may also be compounds known to release in a controlled manner various types of additional ingredients.

The additional ingredients may be dissolved in a solvent of current use in the fields as well known to the person skilled in the art. The solvent is preferably not an alcohol. Examples of such solvents are diethyl phthalate, isopropyl myristate, Abalyn® (rosin resins, available from Eastman), benzyl benzoate, ethyl citrate, limonene or other terpenes, or isoparaffins. Preferably, the solvent is very oil-soluble and highly sterically hindered, like for example Abalyn® or benzyl benzoate. Preferably the additional ingredient comprises less than 30% of solvent. More preferably the additional ingredient comprises less than 20% and even more preferably less than 10% of solvent, all these percentages being defined by weight relative to the total weight of the perfume. Most preferably, the additional ingredient is essentially free of solvent.

According to an embodiment, the emulsion can comprise a co-solvent. According to an embodiment, the co-solvent is a vegetable oil and/or an animal oil.

### Oil-Miscible Co-Solvent:

According to an embodiment, the dispersed phase comprises an oil-miscible co-solvent.

An oil-miscible co-solvent which can be used in the invention may be, for example, tributyl-O-acetylcitrate, triethylcitrate, caprylic triglyceride, triacetin, coconut alkanes (and) coco-caprylate/caprate, propanediol dicaprylate, octanoic acid 1,3-propanediyl ester, isopropyl palmitate, isopropyl myristate, ethyl oleate, triheptanoin, caprylic/capric glycerides, undecane and tridecane, C15-C19 alkanes, squalene, a silicone oil, a glycol ether such as tripropylene glycol methyl ether, dipropylene glycol n-propyl ether, DIPG monomethyl ether, a dimethyl adipate/dimethyl glutarate ester, benzyl benzoate, piperonil butoxyde, coconut oil, or mixtures thereof.

In a preferred embodiment the co-solvent is tributyl-O-acetylcitrate or triethylcitrate.

Preferably the oil-miscible co-solvent chosen in the group consisting of benzyl benzoate, piperonil butoxyde, coconut oil and mixtures thereof in order to get a homogeneous dispersed phase. When present, the co-solvent is preferably used between 5 and 30%, preferably between 10 and 25% by weight based on the total weight of the oil phase.

### Water-Miscible Co-Solvent:

According to an embodiment, the aqueous continuous phase comprises a water-miscible co-solvent, preferably chosen in the group consisting of mono- and polyhydric solvents. Non limiting examples of such solvents can be found from the group containing ethanol, propanol, propylene glycol, hexylene glycol, dipropylene glycol, glycerol, isopropylidene glycerol, butylene glycol (1,3-butanediol), 1,2-butanediol, 2,3-butanediol, 1,2-pentanediol, 1,2-hexanediol, and isopropanol, and mixtures thereof. When present, the co-solvent is preferably used between 5 and 30%, preferably between 10 and 25% by weight based on the total weight of the aqueous phase.

### Stabilizer:

According to the invention, the continuous phase comprises a stabilizer dispersed in water.

According to a particular embodiment, the stabilizer is a molecular emulsifier.

“Molecular emulsifier” are amphiphilic molecules that concentrate at the interface between two phases and modify the properties of that interface. Examples of stabilizers are well known in the art to the skilled person.

According to an embodiment, the molecular emulsifier is a polymeric emulsifier.

The use of polymeric emulsifiers rather than non-ionic emulsifiers is preferred since these stabilizing agents have a higher stability (i.e. charge and steric repulsion) than non-ionic emulsifiers and also prevent aggregation between the solid droplets. Also, non-ionic emulsifiers such as PEG and PPG-based surfactants are non-natural and therefore by excluding this class of molecule from the emulsion of the invention there is no risk of contaminants such as 1,4-dioxane, free glycol ether and free ethylene oxides. Hence a preferred embodiment of the invention is wherein the stabilizer is not a non-ionic emulsifier, preferably the stabilizer is not polyethylene glycol (PEG) or PPG or polyethylene glycol (PEG) or PPG derivatives such as ethoxylated alcohols for example.

A further embodiment is wherein the emulsion of the invention does not comprise polyethylene glycol (PEG) or PPG or polyethylene glycol (PEG) or PPG derivatives such as ethoxylated alcohols for example.

An additional advantage of polymeric emulsifiers is that such molecules are notified as ingredients allowed in minimum risk pesticide products under the minimum risk exemption regulations in 40 CFR 152.25(f) of US EPA and listed in the FIFRA section 25b products, meaning that there are less regulatory obstacles for the commercial exploitation of the emulsion of the invention.

As non-limiting examples, the molecular emulsifier can be chosen in the group consisting of modified starch, gum arabic, pectins, casein, cyclodextrins, lecithins, soy protein, quillaja saponin, and mixtures thereof.

When present, the stabilizer is preferably used between 0.5% and 15%, preferably between 1 and 10% by weight based on the total weight of the emulsion.

A preferred embodiment of the invention is wherein the molecular emulsifier is a polymeric emulsifier and is present at 5% or more by weight based on the total weight of the emulsion, preferably 6%, 7%, 8%, 9% or 10% by weight based on the total weight of the emulsion.

### Water Content

It is important to highlight that the emulsion of the invention comprises more than 50% water, preferably, 60%, 65%, 68%, 70%, 73%, 75%, 80%, 85% or more. This is important since the emulsion is an “oil in water” emulsion and not a “water in oil” emulsion.

Advantages of having more that 50% water include the stability of the emulsion, since more than 50% other components may cause the emulsion to solidify or otherwise become unusable in certain environmental conditions. Furthermore, unit costs can be reduced since water is a low cost commodity.

As can be seen from the accompanying examples, the inventors prepared a series of oil-in-water emulsions of the invention. The emulsions each have over 70% water content. Hence a preferred embodiment of the invention is wherein the emulsion of the invention comprises more than 70% water, preferably 73%, 75%, 80%, 85% or more.

### Optional Ingredients:

The emulsion may also comprise optional ingredients such as a weighting agent (such as estergum, Damar gum, acetyl tributyl citrate), a viscosifier, a gelling agent (such as agar gum, gellan gum, guar gum, tragacanth gum, cellulose derivatives, xanthan gum), pH adjusters, and mixtures thereof.

Examples of viscosifiers which can be used in the present invention include carboxylic acid homopolymer, carboxylic acid copolymers.

According to the present invention, an emulsion composition may further comprise optional other ingredients such as colorants, preservatives, emollients, humectants, antioxidants, free radical scavengers, POV remediation agents, cooling agents, vitamins, fixatives, cosmetic benefit agents, chelators, functional polymers, nutrients, growth media or electrolytes.

Such optional ingredients may represent no more than 10%, 3% w/w, or even 2% w/w, the percentages being relative to the total weight of the emulsion.

Examples of cooling agents which can be used in the present invention include menthol, menthol methyl ether, menthol ethylene glycol carbonate (FEMA GRAS 3805), menthol propylene glycol carbonate (FEMA GRAS 3806), menthyl-N-ethyloxamate, monomenthyl succinate (FEMA GRAS 3810), monomenthyl glutamate (FEMA GRAS 4006), menthoxy-1,2-propanediol (FEMA GRAS 3784), 3-hydroxymethyl p-menthane, menthyl ethoxyhydroxyacetate, 2-(4-ethylphenoxy)-N-(1H-pyrazol-5-yl)-N-(2-thienylmethyl)acetamide, WS23 (2-Isopropyl-N,2,3-trimethylbutyramide), FEMA 3804; WS-3 (N-Ethyl-p-menthane-3-carboxamide), FEMA 3455; WS-5 [Ethyl 3-(p-menthane-3-carboxamido)acetate], FEMA 4309; WS-12 (1R,2S,5R)—N-(4-Methoxyphenyl)-p-menthanecarboxamide, FEMA 4681; WS27 (N-Ethyl-2,2-diisopropylbutanamide), FEMA 4557; N-Cyclopropyl-5-methyl-2-isopropylcyclohexanecarboxamide, FEMA 4693, WS-116 (N-(1,1-Dimethyl-2-hydroxyethyl)-2,2-diethylbutanamide), N-(1,1-Dimethyl-2-hydroxyethyl)2,2-diethylbutanamide, FEMA 4603, Menthoxyethanol, FEMA 4154, N-(4-cyanomethylphenyl)-p-menthanecarboxamide, FEMA 4496; N-(2-(Pyridin-2-yl)ethyl)-3-p-menthanecarboxamide, FEMA 4549; N-(2-Hydroxyethyl)-2-isopropyl-2,3-dimethylbutanamide, FEMA 4602 and (also N-(4-(carbamoylmethyl)phenyl)-menthylcarboxamide, FEMA 4684; (1R,2S,5R)—N-(4-Methoxyphenyl)-p-menthanecarboxamide (WS-12), FEMA 4681; (2S,5R)—N-[4-(2-Amino-2-oxoethyl)phenyl]-p-menthanecarboxamide, FEMA 4684; and N-Cyclopropyl-5-methyl-2-isopropylcyclohexanecarbonecarboxamide, FEMA 4693; 2-[(2-p-Menthoxy)ethoxy]ethanol, FEMA 4718; (2,6-Diethyl-5-isopropyl-2-methyltetrahydropyran, FEMA 4680); trans-4-tert butyl cyclohexanol, FEMA 4724; 2-(p-tolyloxy)-N-(1H-pyrazol-5-yl)-N-((thiophen-2-yl)methyl)acetamide, FEMA 4809; Menthone glycerol ketal, FEMA 3807; Menthone glycerol ketal, FEMA 3808; (−)-Menthoxypropane-1,2-diol; 3-(L-Menthoxy)-2-methylpropane-1,2-diol, FEMA 3849; isopulegol; (+)-cis & (−)-trans p-Menthane-3,8-diol, Ratio˜62:38, FEMA 4053; 2,3-dihydroxy-p-menthane; 3,3,5-trimethylcyclohexanone glycerol ketal; menthyl pyrrolidone carboxylate; (1R,3R,4S)-3-menthyl-3,6-dioxaheptanoate; (1R,2S,5R)-3-menthyl methoxyacetate; (1R,2S,5R)-3-menthyl 3,6,9-trioxadecanoate; (1R,2S,5R)-3-menthyl 3.6,9-trioxadecanoate; (1R,2S,5R)-3-menthyl (2-hydroxyethoxy)acetate; (1R,2S,5R)-menthyl-11-hydroxy-3,6,9-trioxaundecanoate; Cubebol, FEMA 4497; N-(4-cyanomethylphenyl) p-menthanecarboxamide, FEMA 4496; 2-isopropyl-5-methylcyclohexyl 4-(dimethylamino)-4-oxobutanoate, FEMA 4230; N-(4-cyanomethylphenyl) p-menthanecarboxamide, FEMA 4496; N-(2-pyridin-2-ylethyl) p-menthanecarboxamide, FEMA 4549, Menthyl lactate, FEMA 3748; 6-isopropyl-3,9-dimethyl-1,4-dioxaspiro[4.5]decan-2-one, FEMA 4285; N-benzo[1,3] dioxol-5-yl-3-p-menthanecarboxamide; N-(1-isopropyl-1,2-dimethylpropyl)-1,3-benzodioxole-5-carboxamide; N—(R)-2-oxotetrahydrofuran-3-yl-(1R,2S,5R)-p-menthane-3-carboxamide; mixture of 2,2,5,6,6-pentamethyl-2,3,6,6a-tetrahydropentalen-3a(1H)-ol and 5-(2-hydroxy-2-methylpropyl)-3,4,4-trimethylcyclopent-2-en-1-one; (1R,2S,5R)-2-isopropyl-5-methyl-N-(2-(pyridin-2-yl)ethyl)cyclohexanecarboxamide, FEMA4549; (2S,5R)-2-isopropyl-5-methyl-N-(2-(pyridin-4-yl)ethyl)cyclohexanecarboxamide; N-(4-cyanomethylphenyl) p-menthanecarboxamide, FEMA 4496; (1S,2S,5R)—N-(4-(cyanomethyl)phenyl)-2-isopropyl-5-methylcyclohexanecarboxamide; 1/7-isopropyl-4/5-methyl-bicyclo[2.2.2]oct-5-ene derivatives; 4-methoxy-N-phenyl-N-[2-(pyridin-2-yl)ethyl]benzamide; 4-methoxy-N-phenyl-N-[2-(pyridin-2-yl)ethyl]benzenesulfonamide; 4-chloro-N-phenyl-N-[2-(pyridin-2-yl)ethyl]benzenesulfonamide; 4-cyano-N-phenyl-N-[2-(pyridin-2-yl)ethyl]benzenesulfonamid; 4-((benzhydrylamino)methyl)-2-methoxyphenol; 4-((bis(4-methoxyphenyl)-methylamino)-methyl)-2-methoxyphenol; 4-((1,2-diphenylethylamino)methyl)-2-methoxyphenol; 4-((benzhydryloxy)methyl)-2-methoxyphenol, 4-((9H-fluoren-9-ylamino)methyl)-2-methoxyphenol; 4-((benzhydrylamino)methyl)-2-ethoxyphenol; 1-(4-methoxyphenyl)-2-(1-methyl-1H-benzo[d]imidazol-2-yl)vinyl4-methoxybenzoate; 2-(1-isopropyl-6-methyl-1H-benzo[d]imidazol-2-yl)-1-(4-methoxyphenyl)vinyl4-methoxybenzoate; (Z)-2-(1-isopropyl-5-methyl-1H-benzo[d]imidazol-2-yl)-1-(4-methoxy-phenyl)vinyl-4-methoxybenzoate; 3-alkyl-p-menthan-3-ol derivatives; derivatives of fenchyl, D-bornyl, L-bornyl, exo-norbornyl, 2-methylisobornyl, 2-ethylfenchyl, 2-methylbornyl, cis-pinan-2-yl, verbanyl and isobornyl; menthyl oxamate derivatives; menthyl 3-oxocarboxylic acid esters; N alpha-(Menthanecarbonyl)amino acid amides; p-menthane carboxamide and WS-23 analogs; (−)-(1R,2R,4S)-dihydro-umbellulol; p-menthane alkyloxy amides; cyclohexane derivatives; butone derivatives; a mixture of 3-menthoxy-1-propanol and 1-menthoxy-2-propanol; 1-[2-hydroxyphenyl]-4-[2-nitrophenyl-]-1,2,3,6-tetrahydropyrimidine-2-one; 4-methyl-3-(1-pyrrolidinyl)-2[5H]-furanone; and combinations thereof.

Examples of fixatives which can be used in the present invention include, for example, caprylyl alcohol, octanol, butyloctanol, isotridecyl alcohol, hexyldecanol, isocetyl alcohol, isostearyl alcohol, octyldecanol, octyldodecanol, decyltetradecanol, tetradecyloctadecanol, neopentyl glycol diethylhexanoate, PPG-3 myristyl ether, and PPG-20 methyl glucose ether.

According to an embodiment, the emulsion does not contain a sun-block agent, and the emulsion does not function as a sun block.

### Preferred Emulsions of the Invention

The following oil-in-water emulsions were prepared by the inventors and are shown in the accompanying examples. Hence they are preferred embodiments of the invention.

A preferred embodiment of the invention is wherein the emulsion comprises: 8.55% stabilizer (preferably gum arabic); 1.88% pH adjuster (preferably 1.28% sodium citrate dibasic sesquihydrate and 0.6% Sodium citrate monobasic); 0.18% preservative agent (preferably 0.09% sodium benzoate and 0.09% potassium sorbate); 74.9% solvent (preferably water); 0.5% arthropod control agent (preferably 0.25% geraniol and 0.25% lemongrass oil); 1.5% solid lipid material (preferably beeswax); and 12.5% weighting agent (preferably triethylcitrate).

A further preferred embodiment of the invention is wherein the emulsion comprises: 8.65% stabilizer (preferably gum arabic); 1.91% pH adjuster (preferably 1.3% sodium citrate dibasic sesquihydrate and 0.61% Sodium citrate monobasic); 0.18% preservative agent (preferably 0.09% sodium benzoate and 0.09% potassium sorbate); 75.76% solvent (preferably water); 5% arthropod control agent (preferably 1.85% geraniol, 1% clove oil, 1% peppermint oil 0.15% cinnamon oil and 1% thyme oil); 1.5% solid lipid material (preferably beeswax); and 7% weighting agent (preferably triethylcitrate).

A further preferred embodiment of the invention is wherein the emulsion comprises: 9% stabilizer (preferably gum arabic); 1.98% pH adjuster (preferably 1.35% sodium citrate dibasic sesquihydrate and 0.63% Sodium citrate monobasic); 0.18% preservative agent (preferably 0.09% sodium benzoate and 0.09% potassium sorbate); 78.84% solvent (preferably water); 0.5% arthropod control agent (preferably 0.25% geraniol and 0.25% lemongrass oil); 1.5% solid lipid material (preferably beeswax); and 8% weighting agent (preferably triethylcitrate).

A further preferred embodiment of the invention is wherein the emulsion comprises: 9.14% stabilizer (preferably gum arabic); 2.01% pH adjuster (preferably 1.37% sodium citrate dibasic sesquihydrate and 0.64% Sodium citrate monobasic); 0.18% preservative agent (preferably 0.09% sodium benzoate and 0.09% potassium sorbate); 80.09% solvent (preferably water); 0.08% arthropod control agent (preferably prallethrin); 1.5% solid lipid material (preferably beeswax); and 7% weighting agent (preferably triethylcitrate).

### Process for Preparing the Oil-In-Water Emulsion

Another object of the invention is a process for preparing an oil-in water emulsion for use as an arthropod control product by application directly on a target arthropod and/or application to an area of habitation of an arthropod, said process comprising the steps of:
(i) Dispersing an oil phase comprising at least one solid lipid material and at least one oil-soluble active arthropod control agent into a continuous aqueous phase comprising a stabilizer to obtain an oil-in-water emulsion, at a temperature above the melting point of the solid lipid material;
(ii) Cooling the emulsion thus obtained to a temperature below the melting point of the solid lipid material.

According to an embodiment, the melting temperature of the dispersed phase is comprised between 40° C. and 80° C., preferably between 40° C. and 70° C., more preferably 45° C. and 65° C.

Hence an embodiment of the invention is wherein step i) is performed at less than 65° C. An advantage of this embodiment of the invention is there will be less degradation of the active agent than at a temperature above 65° C. Moreover, at a lower temperature more of the active agent will be contained within the solid material matrix and hence not exposed to water or air during storage of the emulsion before use.

In step i), stabilizer(s) and agent forming the aqueous phase are mixed with water to obtain a homogeneous continuous phase. The lipid mixture (solid lipid material and oil-soluble material) is added to the continuous phase at room temperature and the agents are heated up together above the melting point of the solid lipid material prior to emulsification. Alternatively, the lipid mixture can also be heated separately above the melting point of the solid lipid material and then added to the warm water phase prior or during the emulsification step. The emulsification step consists of using any known emulsifying method, such as high shear mechanical mixing, sonication or high-pressure homogenization. Such emulsifying methods are well known to the person skilled in the art.

Advantageously, the emulsion presents a drop size having an average diameter (d₅₀) of between 0.1 to 20 microns, preferably 0.5 to 20 microns, most preferably 0.5 to 10 microns.

Another preferred embodiment of the invention is wherein the process of the invention is used to prepare an emulsion characterised by heterogeneous drop size. Preferably the droplet size is in a range of between 0.1 to 20 microns. That is, the emulsion is not dominated by a droplet having a uniform size but rather the drop sizes are distributed across this size range.

The drop size can be measured via any well-established method that allows measurements which are accurate within an experimental error of 5% at the most and preferably below 1%. Suitable well-established methods use laser diffraction particle size analyser (e.g. Coulter LS 13 320 from Beckman Coulter, Brea, CA, USA). Upon analysis the volume statistics (d_{4,3}) was determined to characterize the emulsion.

In step ii) of the process, the emulsion thus obtained is cooled to a temperature below the melting point of the dispersed phase.

Typically, the cooling step is done by reducing temperature of the emulsion of 5 to 40° C. per hour, preferably of 10 to 25° C. per hour, preferably of 12 to 15° C. per hour.

### Consumer Articles

The invention's emulsions can advantageously be used in different fields, such as pest control industry, home care or agriculture.

Consequently, another object of the present invention is represented by a consumer articles comprising the emulsion as defined above.

The nature and type of the constituents of the consumer articles do not warrant a more detailed description here, which in any case would not be exhaustive, the skilled person being able to select them on the basis of his general knowledge and according to the nature and the desired effect of said articles. These formulations do not warrant a detailed description here which would in any case not be exhaustive. The person skilled in the art of formulating such consumer articles is perfectly able to select the suitable components on the basis of his general knowledge and of the available literature.

According to a particular embodiment, the arthropod control product comprises an arthropod control agent and the consumer article is for arthropod control.

By “arthropod control article” is understood to designate a consumer articles which delivers an efficient arthropod controlling effect (e.g. higher lastingness, lower doses needed, more targeted application or better success rate) to the pest target to which it is applied (e.g. cockroaches, bed bugs, ants, termites, aphids or when the agent is easily transferable nests or refugee). For the sake of clarity, said consumer article is not edible to a target organisms (human, or mammalian pets or livestock) The consumer article can be in the form of a sprayable solution, or a gel/viscous. Ideally, the arthropod control agent is directly sprayed on the arthropods. The arthropods will disseminate the effect of the active agent while moving. In a particular embodiment, space in the vicinity of the targeted pest such as nest, refugee or colony sector may be sprayed, foreseeing that the targeted pest would contact the arthropod control agent that would bind to its cuticle.

According to an embodiment, the emulsion is not transferred from a non-target carrier organism to the target organism or the target site.

The invention will now be further described by way of examples. It will be appreciated that the invention as claimed is not intended to be limited in any way by these examples.

### EXAMPLES

### Example 1: Emulsions According to the Invention (with Geraniol & Lemongrass Oil as Oil-Soluble Arthropod Control Agents)

Water phase preparation: in a 100 ml glass flask, 0.1 g of sodium benzoate, 0.1 g of potassium sorbate, 0.7 g of sodium citrate monobasic and 1.5 g of sodium citrate dibasic sesquihydrate were added to 87.6 g water and solubilized under magnetic stirring. 10 g of gum arabic were then slowly added to the solution and magnetic stirring was continued for 4 hrs to solubilize the ingredients and get a homogeneous phase.

Emulsion 1 preparation: in a 20 ml glass vial, Emulsion 1 was prepared by weighing 0.05 g of geraniol and lemongrass oil [ratio 1:1], 0.15 g of beeswax and 1.25 g of triethylcitrate. The oil phase was heated to 65° C. for 30 minutes to get a liquid and homogenous oil phase. Separately, the water phase was heated at 65° C. and 8.55 g of it were added to the oil phase. The warm solution containing the 2 phases was sheared for 30 seconds using an ultrasonic probe to produce an oil-in-water emulsion. The sample was then immersed in a water bath and left to cool down to room temperature.

Emulsion 2 preparation: in another 20 ml glass vial, Emulsion 2 was prepared by weighing 0.05 g of geraniol and lemongrass oil [ratio 1:1], 0.15 g of beeswax, 1.25 g of triethylcitrate and 8.55 g of water phase. The mix was heated at 65° C. for 30 minutes to get all ingredients liquid. The warm solution containing the 2 phases was sheared for 30 seconds using an ultrasonic probe to produce an oil-in-water emulsion. The sample was then immersed in a water bath and left to cool down to room temperature.

TABLE 1
Emulsion compositions

| | Emulsion 1 | Emulsion 2 | |
|---|---|---|---|
| | Effective Amount of | | |
| Ingredients | Ingredient (% wt) | | Function |
| Gum arabic ¹⁾ | 8.55 | 8.55 | Stabilizer |
| Sodium citrate dibasic | 1.28 | 1.28 | pH adjuster |
| sesquihydrate ²⁾ | | | |
| Sodium citrate | 0.6 | 0.6 | pH adjuster |
| monobasic ³⁾ | | | |
| Sodium benzoate ⁴⁾ | 0.09 | 0.09 | Preservative agent |
| Potassium sorbate ⁵⁾ | 0.09 | 0.09 | Preservative agent |
| Demineralized water | 74.9 | 74.9 | Solvent |
| Geraniol ⁶⁾ | 0.25 | 0.25 | Arthropod control |
| | | | agent |
| Lemongrass oil ⁷⁾ | 0.25 | 0.25 | Arthropod control |
| | | | agent |
| Beeswax ⁸⁾ | 1.5 | 1.5 | Solid lipid material |
| Triethylcitrate ⁹⁾ | 12.5 | 12.5 | Weighting agent |

¹⁾ Superstab ™ gum; origin: Nexira
²⁾ Origin: Sigma Aldrich
³⁾ Origin: Sigma Aldrich
⁴⁾ Origin: Alfa Aesar
⁵⁾ Origin: Alfa Aesar
⁶⁾ Origin: Firmenich
⁷⁾ Origin: Firmenich
⁸⁾ Origin: Aldrich
⁹⁾ Origin: Firmenich

Homogeneous and sprayable emulsions were obtained.

### Example 2: Different Shape and Homogeneity of Droplets According to Example 1

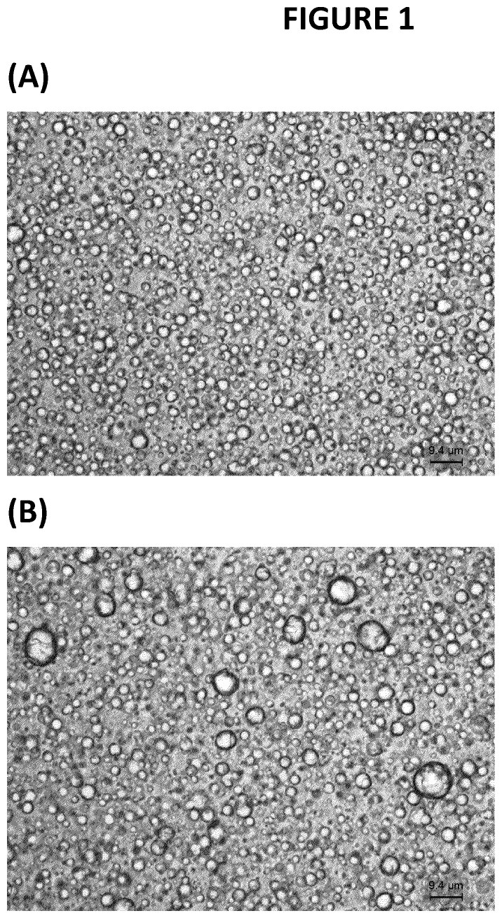

As displayed in FIG. 1, the size and shape of the droplets containing the solid lipid material (i.e. beeswax) differ depending on the formulation process. Indeed, in Emulsion 1, all droplets are of similar size (mean size about 3 m) and would uniformly deliver the active ingredients in a regulated manner (FIG. 1 A). On the other hand, Emulsion 2 contains droplets of different sizes that would allow different delivery rate and longer emission as the larger droplets contain more active than the small ones and will empty more slowly (FIG. 1 B).

It is also to note that the non-spherical aspect of the droplets as well as the high contrast observed at the droplet surface demonstrate their solid/semi-solid physical state within the liquid continuous phase environment. Indeed, as known by a person skilled in the art, a liquid lipidic material emulsified in a liquid water phase with an emulsifier such as gum arabic, would be dispersed as perfectly spherical droplets and would show much less contrast under the same conditions of observation through simple transmitted light microscopy.

### Example 3: Emulsion According to the Invention (with Different Oil-Soluble Arthropod Control Agents)

Water phase preparation: in a 100 ml glass flask, 0.1 g of sodium benzoate, 0.1 g of potassium sorbate, 0.7 g of sodium citrate monobasic and 1.5 g of sodium citrate dibasic sesquihydrate were added to 87.6 g water and solubilized under magnetic stirring. 10 g of gum arabic were then slowly added to the solution and magnetic stirring was continued for 4 hrs to solubilize the ingredients and get a homogeneous phase.

Emulsion 3 preparation: in a 20 ml glass vial, Emulsion 3 was prepared by weighing 0.1 g of clove oil, 0.1 g of mint Piperita oil, 0.185 g of geraniol, 0.015 g of cinnamon oil and 0.1 g of thyme oil. The mix of oils was gently manually shaken before addition of 0.15 g of beeswax, 0.7 g of triethylcitrate and 8.65 g of water phase. The mix was heated at 65° C. for 30 minutes to get all ingredients liquid. The warm solution containing the 2 phases was sheared for 30 seconds using an ultrasonic probe to produce an oil-in-water emulsion. The sample was then immersed in a water bath and left to cool down to room temperature.

Emulsion 4 preparation: in a 20 ml glass vial, Emulsion 4 was prepared by weighing 0.05 g of geraniol and lemongrass oil [ratio 1:1], 0.15 g of beeswax, 0.8 g of triethylcitrate and 9 g of water phase. The mix was heated at 65° C. for 30 minutes to get all ingredients liquid. The warm solution containing the 2 phases was sheared for 30 seconds using an ultrasonic probe to produce an oil-in-water emulsion. The sample was then immersed in a water bath and left to cool down to room temperature.

Emulsion 5 preparation: in a 20 ml glass vial, Emulsion 5 was prepared by weighing 0.008 g of prallethrin, 0.15 g of beeswax, 0.7 g of triethylcitrate and 9.14 g of water phase. The mix was heated at 65° C. for 30 minutes to get all ingredients liquid. The warm solution containing the 2 phases was sheared for 30 seconds using an ultrasonic probe to produce an oil-in-water emulsion. The sample was then immersed in a water bath and left to cool down to room temperature.

TABLE 2
Emulsion compositions

| | Emulsion 3 | Emulsion 4 | Emulsion 5 | |
|---|---|---|---|---|
| Ingredients | Effective Amount of Ingredient (% wt) | | | Function |
| Gum arabic ¹⁾ | 8.65 | 9 | 9.14 | Stabilizer |
| Sodium citrate dibasic | 1.3 | 1.35 | 1.37 | pH adjuster |
| sesquihydrate ²⁾ | | | | |
| Sodium citrate monobasic ³⁾ | 0.61 | 0.63 | 0.64 | pH adjuster |
| Sodium benzoate ⁴⁾ | 0.09 | 0.09 | 0.09 | Preservative agent |
| Potassium sorbate ⁵⁾ | 0.09 | 0.09 | 0.09 | Preservative agent |
| Demineralised water | 75.76 | 78.84 | 80.09 | Solvent |
| Active ingredients (see | 5 | 0.5 | 0.08 | Arthropod control agent |
| Table 3) | | | | |
| Beeswax ⁶⁾ | 1.5 | 1.5 | 1.5 | Solid lipid material |
| | | | | [symbol] |
| Triethylcitrate ⁷⁾ | 7 | 8 | 7 | Weighting agent |

¹⁾ Superstab ™ gum; origin: Nexira
²⁾ Origin: Sigma Aldrich
³⁾ Origin: Sigma Aldrich
⁴⁾ Origin: Alfa Aesar
⁵⁾ Origin: Alfa Aesar
⁶⁾ Origin: Aldrich
⁷⁾ Origin: Firmenich

TABLE 3
Oil-soluble arthropod control agent of the emulsion compositions

| | Emulsion 3 | Emulsion 4 | Emulsion 5 |
|---|---|---|---|
| Oil-soluble arthropod | actives | actives | actives |
| control agent | Effective Amount of active Ingredient (% wt) | | |
| Geraniol ¹¹⁾ | 1.85 | 0.25 | |
| Lemongrass oil ¹²⁾ | | 0.25 | |
| Clove oil ¹³⁾ | 1 | | |
| Peppermint oil ¹⁴⁾ | 1 | | |
| Cinnamon oil ¹⁵⁾ | 0.15 | | |
| Thyme oil ¹⁶⁾ | 1 | | |
| Prallethrin ¹⁷⁾ | | | 0.08 |

8) Origin: Firmenich
9) Origin: Firmenich
10) Clove essential oil; Origin: Firmenich
¹¹⁾ Origin: Firmenich
¹²⁾ Cinnamon essential oil; Origin: Firmenich
¹³⁾ Thyme essential oil; Origin: Firmenich
¹⁴⁾ Origin: Firmenich

A homogeneous and sprayable emulsion was obtained in all cases.

### Example 4: Solutions Made as Comparative Examples

Additional solutions were made as comparative examples by adding a blend of essential oils mentioned in Table 3 directly into water and ethanol and vigorously shaking just prior use, i.e. 0.5% of a mixture of geraniol and lemongrass oil (same active content as in Emulsion 4) in 5% of ethanol and 94.5% of water, referred to as 0.5% GL, and 5% of a mixture of clove oil, peppermint oil, geraniol, cinnamon oil and thyme oil (same active content as in Emulsion 3) in 5% of ethanol and 90% of water, referred to as 5% EO.

### Example 5: Controlling Effect on Cockroaches of the Emulsions According to Examples 1 and 3

Controlling efficacy was tested against nymphs of the American cockroach, Periplaneta americana (L). This species was chosen as it is considered being a human pest carrying many pathogenic human micro-organisms as known by a person skilled in the art. In addition, different species of cockroach exhibit a gregarious behavior by gathering in refuges.

Efficacy of Emulsion 2 described in Example 1 (Table 1) and of Emulsions 3, 4 & 5 described in Example 3 (Table 2) were assessed. Onto each cockroach was sprayed on average 544 mg±9 mg of emulsion according to Examples 1 and 3, resulting in a quantity of 82 mg±3 mg of active and lipid material loaded on the insect, the rest being lost during the spraying and the cockroach movement. It was then directly and individually placed into a closed plastic Petri dish (85 mm) and regularly observed for mortality.

Pure water was used as control samples in all experiments.

As shown in Table 4, the blend of geraniol and lemongrass oils 0.5% GL used at low concentration (0.5%) described in Example 4 was not sufficient to kill any of the cockroaches when diluted in a water-ethanol mixture (19:1). However, the same amount of active ingredients applied on the cockroaches using Emulsion 2 according to the present invention was sufficient to kill the cockroaches in all the three replicates (Table 4). Two were killed within the hour post-application whereas the last one died 18 h after application. To demonstrate the importance of the formulation, Emulsion 4 using a different amount of solvent, only allowed to kill one of the three cockroaches tested (Table 4) 16 h post-application. As already mentioned above, it is worthwhile to note that all insects received a similar amount of active ingredients (Table 4).

TABLE 4
Efficacy of different formulations containing a blend of essential
oils as oil-soluble arthropod control agent at 0.5%

| | | number of | quantity |
|---|---|---|---|
| | number of | dead | loaded on the |
| | replicates | cockroaches | cockroaches |
| control | 2 | 0 | 0 mg |
| 0.5% GL | 3 | 0 | 84 mg |
| Emulsion 2 | 3 | 3 | 79 mg |
| Emulsion 4 | 3 | 1 | 80 mg |

0.5% GL corresponds to the active blend in Emulsion 2 and 4 indicated in example 4, i.e. 0.5% of geraniol + lemongrass oil

As shown in Table 5, a blend of different essential oils used at higher concentration (5%) than the previous example was sufficient to kill the cockroaches when diluted in a water-ethanol mixture (18:1) (5% EO). A knock down effect appears after application, but between 1 h and 8 h was needed to reach death. On the other hand, the same amount of active ingredients applied onto the cockroaches using Emulsion 3 described in the present invention was sufficient to kill the cockroaches in all the three replicates (Table 5) within the first hour post application. Due to the long grooming observed after application of Emulsion 3 compared to the other lotion, one can imagine that the fastest killing effect is linked to the ingestion of the active ingredients loaded within the solid lipid material by the pest. As already mentioned above, it is worthwhile to note that all insects received a similar amount of active ingredients (Table 5).

TABLE 5
Efficacy of different formulations containing a blend of essential
oils as oil-soluble arthropod control agent at 5%

| | | number of | quantity |
|---|---|---|---|
| | number of | dead | loaded on the |
| | replicates | cockroaches | cockroaches |
| control | 2 | 0 | 0 mg |
| 5% EO | 2 | 2 | 87 mg |
| Emulsion 3 | 3 | 3 | 81 mg |

5% EO corresponds to the active blend in Emulsion 3 indicated in example 4, i.e. 5% of essential oils

As shown in Table 6, Emulsion 5 described in the present invention was sufficient to kill the cockroaches in all replicates. Whereas prallethrin is a strong killing agent, and due to the lower diffusion through the solid lipid material in the emulsions of the present invention, it took >12 h to kill the cockroaches. In nature, this time laps would have allowed the cockroach to move back to its refuge and spread the poison within its community.

TABLE 6
Efficacy of a formulation containing prallethrin

| | | number of | quantity |
|---|---|---|---|
| | number of | dead | loaded on the |
| | replicates | cockroaches | cockroaches |
| control | 2 | 0 | 0 mg |
| Emulsion 5 | 2 | 2 | 82 mg |

### Example 6: Emulsion According to the Invention (with Geraniol & Lemongrass Oil as Oil-Soluble Arthropod Control Agent)

Water phase preparation: in a 100 ml glass flask, 0.1 g of sodium benzoate, 0.1 g of potassium sorbate, 0.7 g of sodium citrate monobasic and 1.5 g of sodium citrate dibasic sesquihydrate were added to 87.6 g water and solubilized under magnetic stirring. 10 g of gum arabic were then slowly added to the solution and the magnetic stirring was let for 4 hrs to solubilize the ingredients and get a homogeneous phase.

Emulsion 6 preparation: in a 20 ml glass vial, Emulsion 6 was prepared by weighing 0.05 g of geraniol and lemongrass oil [ratio 1:1], 0.15 g of beeswax, 0.6 g of tributyl-O-acetylcitrate and 9.2 g of water phase. The mix was heated at 65° C. for 30 minutes to get all ingredients liquid. The warm solution containing the 2 phases was sheared for 30 seconds using an ultrasonic probe to produce an oil-in-water emulsion. The sample was then immersed in a water bath and left to cool down to room temperature.

TABLE 7
Composition of Emulsion 6

| | Emulsion 6 | |
|---|---|---|
| Ingredients | Effective Amount (% wt) | Function |
| Gum arabic ¹⁾ | 9.2 | Stabilizer |
| Sodium citrate dibasic | 1.38 | pH adjuster |
| sesquihydrate ²⁾ | | |
| Sodium citrate | 0.64 | pH adjuster |
| monobasic ³⁾ | | |
| Sodium benzoate ⁴⁾ | 0.09 | Preservative |
| | | agent |
| Potassium sorbate ⁵⁾ | 0.09 | Preservative |
| | | agent |
| Demineralized water | 80.60 | Solvent |
| Geraniol ⁶⁾ | 0.25 | Arthropod |
| | | control agent |
| Lemongrass oil ⁷⁾ | 0.25 | Arthropod |
| | | control agent |
| Beeswax ⁸⁾ | 1.5 | Solid lipid |
| | | material |
| Tributyl-O- | 6 | Weighting agent |
| acetylcitrate ⁹⁾ | | |

¹⁾ Superstab ™ gum; origin: Nexira
²⁾ Origin: Sigma Aldrich
³⁾ Origin: Sigma Aldrich
⁴⁾ Origin: Alfa Aesar
⁵⁾ Origin: Alfa Aesar
⁶⁾ Origin: Firmenich
⁷⁾ Origin: Firmenich
⁸⁾ Origin: Aldrich
⁹⁾ Origin: Aldrich

Homogeneous and sprayable emulsion was obtained.

### Example 7: Modification of the Behavior of Cockroaches Due to Emulsions Releasing Essential Oils as Oil-Soluble Arthropod Control Agent

Controlling efficacy was tested against nymphs of the American cockroach, Periplaneta americana (L). This species was chosen as it is considered as a human pest by a person skilled in the art carrying many pathogenic human microorganisms and viruses. In addition, different species of cockroach exhibit a gregarious behavior by gathering in refuges. The experiment was carried out with nymphs to avoid any confounding effect linked to sexual behavior.

Efficacy of Emulsions 2, 4 and 6 described in Example 1 (Table 1), Example 3 (Table 2) and Example 6 (Table 7) respectively, were assessed. In addition, the blend 0.5% GL described in Example 4 was used as a control of active ingredients without the solid lipid material. A commercially available product, Zevo, containing similar active ingredients level, i.e. 0.25% of geraniol and 0.25% of lemongrass oil, solubilized in a mixture of white mineral oil, isopropyl alcohol, butyl lactate, isopropyl myristate and triethyl citrate as inert carrier materials, was also assessed.

In that experiment, one cockroach was marked prior to being sprayed with the testing product or a sham operation. This treated cockroach was then placed into a plastic arena (260 mm diam) already containing two other untreated cockroaches. The interactions between the three cockroaches were filmed and the number of intentional contacts between each individual was reported during 30 minutes after introduction of the treated cockroach.

In the first experiment, the number of intentional contacts towards untreated cockroaches were measured; how many times does the treated or the untreated cockroaches manage to get into contact with an untreated cockroach. As displayed in Table 8, in the control experiment without any treatment, the marked and sham treated cockroach elicited only 6% less contact (18.25) compared to the number of contacts between the two untreated cockroaches (19.5). This number of intentional contacts was decreased after treatment with the blend of active ingredients (Table 8). Indeed, when the cockroach was treated with the active ingredients but without the solid lipid material (i.e. 0.5% GL), the number of contacts between the treated and untreated (10.5) but also between untreated cockroaches (10.3) was reduced (Table 8). Nevertheless, like for the control there was still no difference between the number of intentional contacts between the two groups (+2% for treated cockroach). In contrast, when the cockroach was treated with the active ingredients loaded within the solid lipid material (i.e. Emulsions 2, 4, and 6), there were drastically less contacts (−73%) between the treated cockroach and an untreated cockroach (2.8) than between the two untreated cockroaches (10.4; Table 8). To conclude, the presence of the active ingredient on an individual in the arena tends to decrease the number of interactions between the cockroaches, treated or not. In addition, treated cockroaches with the loaded solid lipid material have a lower probability of contacting untreated cockroaches when they are carrying the active ingredients causing even more communication disruption within the community.

TABLE 8
Measure of intentional contact between treated cockroach towards
untreated cockroach and between untreated cockroaches

| | Contact of Untreated | Contact of Treated |
|---|---|---|
| | towards Untreated | towards Untreated |
| control | 19.5 | 18.3 |
| 0.5% GL | 10.3 | 10.5 |
| Emulsion 2 | 11.0 | 0 |
| Emulsion 6 | 22.5 | 2.5 |
| Emulsion 4 | 6.0 | 0.5 |

In a similar experiment, the interactions elicited by the two untreated cockroaches present in the arena were individually assessed; how many intentional contacts did they elicit towards the other untreated cockroach and towards the treated cockroach. As displayed in Table 9, in the control experiment the untreated cockroach made a similar ratio of contact with the marked and sham treated cockroach (57.9%) than with the other untreated cockroach (42.1%). When the cockroach was treated with the active ingredient in the absence of the solid lipid material, it was less contacted by the untreated cockroaches i.e. −24% and −22% for 0.5% GL and Zevo solutions respectively. (Table 9). But this decrease was even more drastic when the active ingredients were loaded within the solid lipid material according to the invention with a mean of 12.8%±7.4% of contact of the untreated cockroach towards the treated cockroach, equivalent to a 46% decrease in contact (Table 9). To conclude, the presence of the active ingredient on an individual in the arena leads to a decrease of the direct contact of the rest of the group towards this individual, disturbing communication within the community. This effect was even more obvious when the active ingredients were loaded within the solid lipid material according to the invention.

TABLE 9
Ratio of contact of Untreated cockroaches
towards Untreated or Treated cockroaches

| | Contact of Untreated | Contact of Untreated |
|---|---|---|
| | towards Untreated | towards Treated |
| control | 42% | 58% |
| 0.5% GL | 66% | 34% |
| Emulsion 2 | 74% | 26% |
| Emulsion 6 | 89% | 11% |
| Emulsion 4 | 100% | 0% |
| Zevo | 64% | 36% |

### Example 8: Emulsions According to the Invention (without Oil-Soluble Arthropod Control Agents)

Water phase preparation: in a 100 ml glass flask, 0.1 g of sodium benzoate, 0.1 g of potassium sorbate, 0.7 g of sodium citrate monobasic and 1.5 g of sodium citrate dibasic sesquihydrate were added to 87.6 g water and solubilized under magnetic stirring. 10 g of gum arabic were then slowly added to the solution and the magnetic stirring was let for 4 hrs to solubilize the ingredients and get a homogeneous phase.

Emulsion 7 preparation: in a 20 ml glass vial, Emulsion 7 was prepared by weighing 0.15 g of beeswax, 1.25 g of triethylcitrate and 8.6 g of water phase. The mix was heated at 65° C. for 30 minutes to get all ingredients liquid. The warm solution containing the 2 phases was sheared for 30 seconds using an ultrasonic probe to produce an oil-in-water emulsion. The sample was then immersed in a water bath and left to cool down to room temperature.

Emulsion 8 preparation: in a 20 ml glass vial, Emulsion 8 was prepared by weighing 0.15 g of beeswax, 0.7 g of triethylcitrate and 9.15 g of water phase. The mix was heated at 65° C. for 30 minutes to get all ingredients liquid. The warm solution containing the 2 phases was sheared for 30 seconds using an ultrasonic probe to produce an oil-in-water emulsion. The sample was then immersed in a water bath and left to cool down to room temperature.

TABLE 10
Emulsion composition without oil-soluble arthropod control agents

| | Emulsion 7 | Emulsion 8 | |
|---|---|---|---|
| | Effective Amount of | | |
| Ingredients | Ingredient (% wt) | | Function |
| Gum arabic ¹⁾ | 8.6 | 9.15 | Stabilizer |
| Sodium citrate dibasic | 1.29 | 1.37 | pH adjuster |
| sesquihydrate ²⁾ | | | |
| Sodium citrate | 0.6 | 0.64 | pH adjuster |
| monobasic ³⁾ | | | |
| Sodium benzoate ⁴⁾ | 0.09 | 0.09 | Preservative agent |
| Potassium sorbate ⁵⁾ | 0.09 | 0.09 | Preservative agent |
| Demineralized water | 75.33 | 80.16 | Solvent |
| Beeswax ⁶⁾ | 1.5 | 1.5 | Solid lipid material |
| Triethylcitrate ⁷⁾ | 12.5 | 7 | Weighting agent |

¹⁾ Superstab ™ gum; origin: Nexira
²⁾ Origin: Sigma Aldrich
³⁾ Origin: Sigma Aldrich
⁴⁾ Origin: Alfa Aesar
⁵⁾ Origin: Alfa Aesar
⁶⁾ Origin: Aldric
⁷⁾ Origin: Firmenich

An homogeneous and sprayable emulsion was obtained.

### Example 9: Modification of the Behavior of Cockroaches Due to Solid Lipid Material

The testing protocol was the same as in Example 7.

Efficacy of Emulsions 1, 4, 5, 6, 7 and 8 described in Example 1 (Table 1), Example 3 (Table 2), Example 6 (Table 7), and Example 8 (Table 10), were assessed. In addition, the blend 0.5% GL displayed in Example 4 was used as control of active ingredients without the solid lipid material.

In that experiment, the activity of each of the three cockroaches present in the arena was timely measured for 30 minutes after the introduction of the treated cockroach. Therefore, the percentage of time spent walking, resting or grooming was quantified for each individual.

In the first experiment, the activity of the treated cockroach was measured. As displayed in Table 11, in the control experiment the marked and sham treated cockroach (control) spent 43% of time walking on the ground and 57% resting. Applying an active repellent ingredient (0.5% GL) does not modify much this activity pattern: with only a mean 3% change in moving and resting activities (Table 11). On the other hand, applying the same active ingredient loaded within the solid lipid material drastically decreased the time spent walking for the treated cockroach while the time spent grooming drastically increased (+21%) with Emulsions 1, 4 and 6 (Table 11). But a similar effect was also obtained using Emulsion 7 (+17%) that is made of solid lipid material only loaded with the solvent triethyl citrate and in the absence of oil-soluble arthropod control agents (Table 11). We can therefore conclude that the solid lipid material sprayed onto the cockroach increases its grooming time. It reinforced the interest of the solid lipid material displayed in this invention as a good method to have the controlling agent ingested by the cockroach.

TABLE 11
Percentage of activity of the treated cockroaches

| | | 0.5% | Emul- | Emul- | Emul- | Emul- |
|---|---|---|---|---|---|---|
| | control | GL | sion 1 | sion 6 | sion 4 | sion 7 |
| stop | 57% | 60% | 77% | 76% | 81% | 74% |
| walk | 43% | 40% | 23% | 24% | 19% | 26% |

In the second experiment, the activity of the untreated cockroaches was measured. As displayed in Table 12, in the control experiment the untreated cockroach spent 50% of time walking on the ground and 50% resting. This pattern is similar to the marked and sham treated cockroach with a mean activity change of 3% (Tables 11 & 12). Applying active repellent ingredients onto a cockroach modified the activity pattern of the untreated cockroaches by increasing their walking activities (Table 12). Indeed, when a cockroach treated with 0.5% GL or Emulsion 6 was present in the arena, the movement of the untreated cockroaches increased by +12% and +14% respectively (Table 12). On the other hand, emulsions not containing volatile repellent active ingredients, but only solid lipid material loaded with solvent (Emulsion 8) or an insecticide (Emulsion 5) led to a very similar activity pattern of the untreated cockroaches as the control with 3% and 0% more movement respectively (Table 12). In conclusion, this experiment demonstrated the relevant release of volatile actives from solid lipid material of the emulsion presented in the present invention.

TABLE 12
Percentage of activity of the untreated cockroaches

| | stop | walk |
|---|---|---|
| control | 50% | 50% |
| 0.5% GL | 38% | 62% |
| Emulsion 6 | 36% | 64% |
| Emulsion 8 | 53% | 47% |
| Emulsion 5 | 50% | 50% |

### Example 10: Modification of the Behavior of Mealworm Beetles Due to Deposition of Solid Lipid Material on an Area

Mealworm beetles, Tenebrio molitor (L), are considered as human pests because larvae and adults feed on stored food. They breed prolifically and can gather in large populations, especially in protected areas such as henhouses in which they cause huge damage. Due to their status of non-model organism, as known by a person skilled in the art Tenebrio molitor is useful in making proof of concept studies.

In this assay, an emulsion according to the invention was applied (544 mg±9 mg) on half of a plastic Petri dish (85 mm) transformed in an arena. This side was named Test side while the other half side was kept without product and served as refuge for the arthropod (Controlside). One insect at a time was deposited on the control side and its activity was recorded and observed for two hours, i.e. time spent walking or not moving in each half of the Petri dish.

Efficacy of Emulsion 3 described in Example 3 (Table 2) and of Emulsion 8 described in Example 8 (Table 10) were assessed. As controls, the solution 5% EO described in Example 4 and detailed in Table 3 as well as water were also applied to the arena test side. It is important to note that Emulsion 3 contains the solid lipid material as in Emulsion 8 but loaded with the active ingredients of solution 5% EO. Comparison of the movement of the mealworm beetles between the two halves of the arena was carried out.

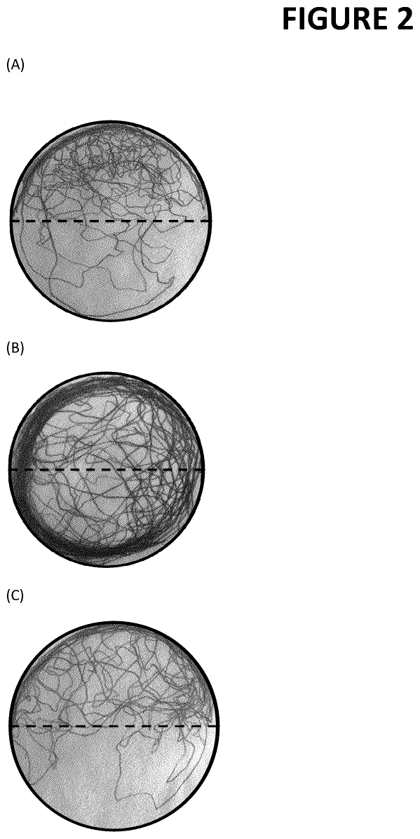

During the first experiment, the mealworm beetle displacement inside the arena was tracked and reported in FIG. 2. The avoidance of the test side treated with either 5% EO or Emulsion 3 formulations can be clearly observed; the mealworm beetle mostly walking in the control side (FIGS. 2A & C). On the other hand, the tracking path of the mealworm beetle recorded when the test side was treated with water demonstrated the similarity between the two halves of the arena (FIG. 2 B).

This result was confirmed by quantifying the time spent walking in both sides of the arena. Indeed, as displayed in Table 13, mealworm beetles spent similar amounts of time between the test side (52%) and the control side (48%) of the arena when the test side was treated with water. As expected, this amount of time spent in the test side decreased by 46% and 43% when the test side was treated with 5% EO or with Emulsion 3 respectively. Interestingly, Emulsion 8, that corresponds to Emulsion 3 but without the oil-soluble arthropod control agent, did not decrease the time spent walking by the mealworm beetle inside the test side, but even seems to attract it by 20% more time spent on that test side compared to the control side treated with water (Table 13). It is also important to note that despite having a lower release of active ingredients from Emulsion 3 compared to free oils of the 5% EO formulation, the efficacy was equivalent but should last longer with Emulsion 3 due to solid lipid material retention properties displayed in the present invention.

TABLE 13
Percentage of time spent moving by the mealworm
beetle inside the arena partially treated

| | Control side | Test side |
|---|---|---|
| control | 48% | 52% |
| Emulsion 8 | 28% | 72% |
| 0.5% GL | 94% | 6% |
| Emulsion 3 | 91% | 9% |

### Example 11: Modification of the Behavior of Mealworm Beetles Due to Deposition of Solid Lipid Material on an Area

The testing protocol was the same as in Example 10.

Efficacy of Emulsion 1 described in Example 1 (Table 1) and of Emulsion 6 described in Example 6 (Table 7) were assessed. As controls, the solution 0.5% GL displayed in Example 4 as well as water were also applied to the arena test side. It is important to note that both Emulsion 1 and Emulsion 6 as well as 0.5% GL contain the same amount of oil-soluble arthropod control agent described in Table 3. The differences between these solutions are the solvent loaded with active ingredients in the solid lipid material, i.e. triethyl citrate in Emulsion 1 and tributyl-O-acetylcitrate in Emulsion 6, and the absence of solid lipid material in 0.5% GL. A comparison of the mealworm activity and of their movement between the two halves of the arena was made.

Regarding mealworm beetle activity, the time spent moving and not moving inside the arena was quantified. As displayed in Table 14, when the test side has been treated with water the arthropod spent almost the full testing session moving inside the arena with only 18% of time spent non-moving. On the other hand, when the active ingredient was present in the arena, the arthropod spent drastically less time moving (−58%), demonstrating more freezing behavior (Table 14). Emulsion 6 followed by Emulsion 1 and then 0.5% GL triggered less movement of the mealworm beetle with a reduction of 69%, 55% and 51% respectively (Table 14). We can conclude that the presence of the active ingredients in the arena increased the freezing behavior of the mealworm beetles. In addition, formulation of the active ingredients influences this behavior, probably due to additional time spent grooming by the mealworm beetles.

TABLE 14
Activity of the mealworm beetle inside the arena partially treated

| | Control side | Test side |
|---|---|---|
| control | 18% | 82% |
| 0.5% GL | 69% | 31% |
| Emulsion 1 | 73% | 27% |
| Emulsion 6 | 87% | 13% |

In a subsequent experiment, the time spent walking inside both sides of the arena was quantified. As already displayed in Table 13, there is a clear trend of spending more time in the control side (+26%) when the tested side is treated with active ingredients compared to a treated side treated with water (Table 15). Indeed, 19%, 40% and 19% less time were spent in the test side treated with 0.5% GL, Emulsion 1 and Emulsion 6 respectively compared to the control (Table 15).

One may notice the lower repellent efficacy of arthropod control agents between Examples 10 and 11 that can be straightforwardly linked to the identity of active ingredients and above all the concentration applied, i.e. 5% of active in Example 10 vs 0.5% in Example 11. Therefore, the same conclusions can be reached between these two experiments, demonstrating the interest of the oil-in-water emulsion displayed in this invention.

TABLE 15
Percentage of time spent moving by the mealworm
beetle inside the arena partially treated

| | Control side | Test side |
|---|---|---|
| control | 48% | 52% |
| 0.5% GL | 67% | 33% |
| Emulsion 1 | 88% | 12% |
| Emulsion 6 | 67% | 33% |

### Example 12: Modification of the Behavior of Cockroach Due to Deposition of Solid Lipid Material on an Area

The testing protocol was the same as in Example 10. However, Tenebrio molitor (L) was replaced by Periplaneta americana (L) as target organism. In addition, the goal of this experiments was primarily to assess the killing efficacy of an active loaded in the solid lipid material while it was not directly sprayed onto the targeted arthropod within its close environment.

For this experiment, water and Emulsion 8 (described in Table 10 of Example 8) used as controls were compared to the oil-in-water emulsion of prallethrin (Emulsion 5) described in Table 2 of Example 3). Each stimulus was sprayed on one half of the arena, while the other half was left untreated as refuge. Cockroach activity was observed, and time spent on each side of the arena was measured.

Knock down effect, that a person skilled in the art would define as partial paralysis, happened after 34 minutes in presence of Emulsion 5. Before that, the cockroach spent about 20 minutes in the control side and about 14 minutes in the test side of the arena. It died a few hours later.

On the other hand, cockroaches placed into the arena with the test side treated with water or Emulsion 8 spent respectively 35% and 33% walking in the test side and were still alive several days after the end of the experiments.

This result demonstrated the efficacy of transmitting the control agent loaded in the solid lipid material from the substrate onto the body of the cockroach on which it acts efficiently.

### Example 13: Demonstration of the Cuticle Binding Properties of the Solid Lipid Material

The goal of this experience was to visually display the presence of the active on the arthropod cuticle. Two distinct parts of arthropods were selected as example: the tarsi of Periplaneta americana (L) and the elytron of Tenebrio molitor (L). Emulsion 2 described in Example 1 and formulation 0.5% GLf described in Example 4 described below were sprayed onto the insect prior of being examined under a confocal microscope (Leica TCS SP8; Objective ×20; Excitation wavelength 552 nm).

In order to visualize more precisely the position of the active ingredients, 3 mg/ml of fluorescent agent (Nile Red from Fluka) was added to triethylcitrate and Emulsion 2 was prepared as described in Example 1. Similarly, 3 mg/ml of Nile Red was added to the oil phase in formulation 0.5% GL described in Example 4.

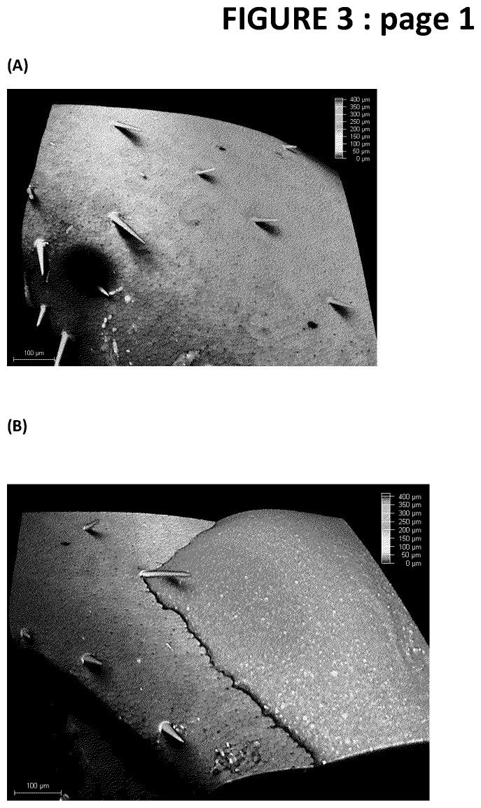

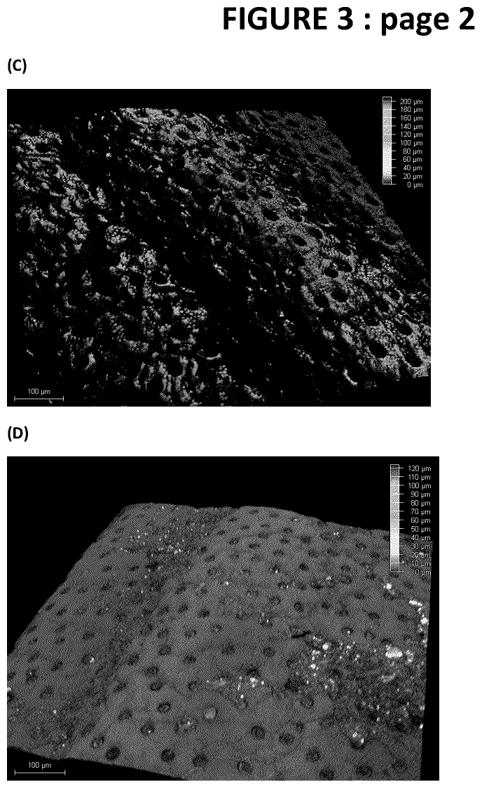

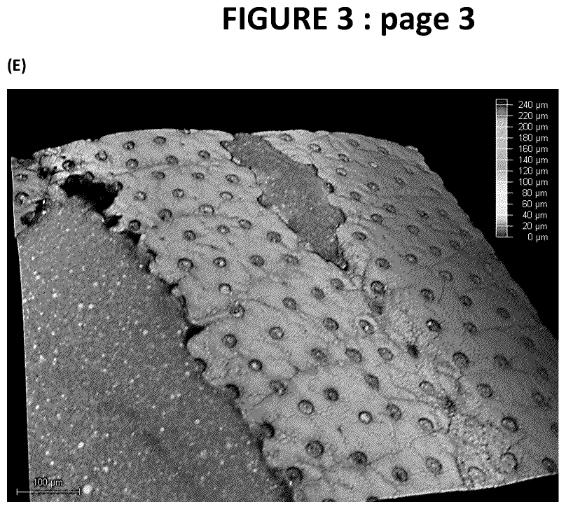

As displayed in the images (FIGS. 3A & B), the oil-in-water emulsion is clearly visible on the cuticle of the cockroach, forming a clear overlaying cluster of fluorescent solid lipid materials (white spots on the right of the FIG. 3B). The evaporation of the water from the oil-in-water emulsion would leave the solid lipid material loaded with the active arthropod agent, acting as a reservoir for slow diffusion of the active through the cuticle, and for potential ingestion of the active by the arthropod and others in the nest.

It is to note the size of the solid lipid materials compared to the sensilla of the cockroach leg.

As displayed in the images (FIGS. 3C & E), the oil-in-water emulsion is clearly visible on the cuticle of the mealworm beetle, forming a clear overlaying cluster of fluorescent solid lipid materials (white spots on the left and top central of the FIG. 3 E). On the other hand, images in FIGS. 3C & D also allows display the presence of the hydrophobic active ingredients at the surface of the mealworm beetle cuticle after using the 0.5% GLf Example 4 solution. But when comparing the images in FIGS. 3D & E, one can clearly see the more diffusive, spread and weak fluorescence after spraying the 0.5% GLf (FIG. 3D) compared to the well-marked and distributed fluorescence when spraying the oil-in-water Emulsion 2.

1. An oil-in-water emulsion comprising:
a dispersed oil phase comprising at least one solid lipid material and at least one oil-soluble arthropod control agent, and
a continuous aqueous phase comprising a stabilizer,
wherein said emulsion is configured for use as an arthropod control product by application directly on a target arthropod and/or application to an area of habitation of an arthropod.

2. The oil-in-water emulsion according to claim 1, wherein the amount of the oil-soluble arthropod control agent is between 0.01% and 25% by weight, based on the total weight of the emulsion.

3. The oil-in-water emulsion according to claim 1, wherein the amount of solid lipid material is comprised between 1 to 50% by weight based on the total weight of the oil phase.

4. The oil-in-water emulsion according to claim 1, wherein the arthropod control agent is selected from the group consisting of ethyl 3-(acetyl(butyl)amino)propanoate (IR3535®), N,N-diethyl-3-methylbenzamide (DEET), p-menthane-3,8-diol (PMD), Eucalyptus citriodora oil, Citronella spp. oil, sec-butyl 2-(2-hydroxyethyl)piperidine-1-carboxylate (picaridin), vanillin, Castor oil, Cedarwood oil, Cinnamon oil, citronellal, Clove oil, Corn oil, Cornmint, Cornmint oil, Cottonseed oil, 4-Allyl-2-methoxyphenol (Eugenol), Garlic oil, (2E)-3,7-Dimethylocta-2,6-dien-1-ol (Geraniol), Geranium oil, Lemongrass oil, Linseed oil, Peppermint, Peppermint oil, 2-Phenylethyl propionate, Rosemary oil, Sesame oil, Soybean oil, Spearmint, Spearmint oil, Thyme oil, Mint, Mint oil, Pepper extract, Wintergreen oil, Lavender oil, Lavandula hybrida ext., Lavandin oil, Lemon oil, Margosa extract, Mentha arvensis ext., Metofluthrin, Nonanoic acid, Pyrethrins and Pyrethroids, 2,3,4,5-bis(butyl-2-ene)tetrahydrofurfural (MGK Repellent 11), cineole, cinnamaldehyde, citral, citronellol, coumarin, dibutyl phthalate, diethyl phthalate, dimethyl anthranilate, dimethyl phthalate, ethyl vanillin, Eucalyptus oil, nootkatone, delta-octalactone, delta-nonalactone, delta-decalactone, delta-undecalactone, delta-dodecalactone, gamma-octalactone, gamma-nonalactone, gamma-decalactone, gamma-undecalactone, gamma-dodecalactone, hydroxy citronellal, lime oil, limonene, linalool, methyl anthranilate, Mint spicata, myrcene, Neem oil, sabinene, 0-caryophyllene, (1H-indol-2-yl)acetic acid, anethole, Anise oil, Basil oil, Bay oil, camphor, ethyl salicylate, Evergreen oils, Pine oil, tetramethrin, allethrin, (RS)-α-cyano-3phenoxybenzyl-(1RS)-cis, cypermethrin, prallethrin, acetamiprid, azadirachtin, bendiocarb, bifenthrin, chlorpyrifos, deltamethrin, diazinon, dichlorvos, fipronil, imidacloprid, linalool, malathion, Margosa extract, nicotine, permethrin, rotenone, S-methoprene, spinosad (Spinosyn A), spinosyn D, transfluthrin, anisic alcohol, octahydrocoumarin, (+−)-2,5-dimethyl-2-indanmethanol, 4,4A,5,9B-tetrahydro-indeno[1,2-D]-1,3-dioxin, 2,4-dimethyl-4,4a,5,9b-tetrahydroindeno[1,2-d][1,3]dioxine, entomopathogenic bacterias, Bacillaceae, Burkholderia, Chromobacterium, Pseudomonas, Saccharopolyspora, Serratia, Streptomyces, Yersinia, Xenorhabdus and Photorhabdus species, entomopathogenic fungi, Metarhizium anisopliae, Beauveria bassiana, Hirsutella, Isaria, Lecanicillium, Paecilomyces and Verticillium species, entomopathogenic virus, entomopathogenic nematodes, Steinernema and Heterorhabditis species, yeasts, specifically designed RNAi, enzymes, chitiniase, semiochemicals or pheromones, stimuli eliciting sexual attraction, (periplanetone B and gentisyl quinone isovalerate of the cockroaches Periplaneta americana (L) and Blattella germanica (L) respectively, (E,Z,Z)-3,8,11-Tetradecatrienyl acetate of the tomato leaf miner Tuta absoluta (Meyrick), or stimuli eliciting aggregation, (verbenone used by the bark beetles like Dendroctonus ponderosae (Hopkins) as an anti-aggregation pheromone or Z,E-alpha-farnesene used by the fire ants Atta geminata (Fabricius) to recruit and as an orientation pheromone, stimuli eliciting identification of species, (olefins and other hydrocarbons of termites, olefins and other hydrocarbons of Cryptotermes brevis (Walker), C. cynocephalus (Light), Procryptotermes corniceps (Snyder) or Neotermes connexus (Snyder), or stimuli eliciting identification of colonies or nestmates, specific components of the cuticular hydrocarbon profile by wasps, specific components of the cuticular hydrocarbon profile by Polistes dominulus (Christ), or stimuli eliciting information on development stage, (oxo-aldehydes of the bed bugs Cimex lectularius (L), or stimuli eliciting a message, (E-β-farnesene and dimethylpyrazine used as alarm pheromones by aphids such as Phorodon humuli (Schrank) and the fire ants A. geminate (F) respectively, and mixtures thereof.

5. The oil-in-water emulsion according to claim 1, wherein the continuous phase comprises a hydrophilic active ingredient.

6. The oil-in-water emulsion according to claim 1, wherein the solid lipid material is selected from the group consisting of a non-vegetable glyceride, vegetable glyceride, a non-vegetable wax, a vegetable wax, beeswax, carnauba wax, palm stearin, jojoba wax, and mixtures thereof.

7. The oil-in-water emulsion according to claim 1, wherein the emulsion comprises an oil-miscible co-solvent.

8. The oil-in-water emulsion according to claim 1, wherein the emulsion comprises a water-miscible co-solvent.

9. The oil-in-water emulsion according to claim 1, wherein the stabilizer is selected from the group consisting of Gum Arabic, Pectins, Casein, cyclodextrins, lecithins, soy protein, quillaja saponin, silica, calcium carbonate, zinc oxide, and mixtures thereof.

10. The oil-in-water emulsion according to claim 1, wherein it comprises an additional component selected from the group consisting of a weighting agent, a viscosifier, a gelling agent, and mixtures thereof.

11. A process for preparing an oil-in water emulsion configured for use as an arthropod control product, said process comprising the steps of:
dispersing an oil phase comprising at least one solid lipid material and at least one oil-soluble arthropod control agent into a continuous aqueous phase comprising a stabilizer to obtain an oil-in-water emulsion, at a temperature above the melting point of the solid lipid material; and
cooling the emulsion thus obtained to a temperature below the melting point of the solid lipid material.

12. The process according to claim 11, wherein the melting point of the solid lipid material is between 40° C. and 80° C.

13. A consumer article comprising the emulsion according to claim 1.

14. The consumer article according to claim 13, wherein it is in the form of a sprayable solution, or a gel/viscous lotion.

15. The oil-in-water emulsion according to claim 1, wherein the amount of solid lipid material is comprised between 5 to 20% by weight based on the total weight of the oil phase.

16. The oil-in-water emulsion according to claim 1, wherein the emulsion comprises an oil-miscible co-solvent selected from the group consisting of tributyl-O-acetylcitrate, triethylcitrate, caprylic triglyceride, triacetin, coconut alkanes (and) coco-caprylate/caprate, propanediol dicaprylate, octanoic acid 1,3-propanediyl ester, isopropyl palmitate, isopropyl myristate, ethyl oleate, triheptanoin, caprylic/capric glycerides, undecane and tridecane, C15-C19 alkanes, squalene, a silicone oil, a glycol ether, tripropylene glycol methyl ether, dipropylene glycol n-propyl ether, DIPG monomethyl ether, a dimethyl adipate/dimethyl glutarate ester, benzyl benzoate, piperonil butoxyde, coconut oil, and mixtures thereof.

17. The oil-in-water emulsion according to claim 1, wherein the emulsion comprises a water-miscible co-solvent selected from the group consisting of ethanol, propanol, propylene glycol, hexylene glycol, dipropylene glycol, glycerol, isopropylidene glycerol, butylene glycol (1,3-butanediol), 1,2-butanediol, 2,3-butanediol, 1,2-pentanediol, 1,2-hexanediol, and isopropanol, and mixtures thereof.

18. The process according to claim 11, wherein the melting point of the solid lipid material is between 40° C. and 70° C.

19. The process according to claim 11, wherein the melting point of the solid lipid material is between 45° C. and 65° C.
